(19) United States
(12) Patent Application Publication
(10) Pub. No.: US 2013/0312136 A1
(43) Pub. Date: Nov. 21, 2013

(54) Methods and Compositions for Modulating Gene Expression in Plants

(71) Applicants: SYNGENTA PARTICIPATIONS AG, Basel (CH); THE UNIVERSITY OF GUELPH, Guelph (CA)

(72) Inventors: Christophe Liseron-Monfils, Guelph (CA); Manish N. Raizada, Guelph (CA); Gregory Downs, Guelph (CA); Lewis N. Lukens, Guelph (CA); Tong Zhu, Research Triangle Park, NC (US)

(21) Appl. No.: 13/891,952

(22) Filed: May 10, 2013

Related U.S. Application Data
(60) Provisional application No. 61/649,757, filed on May 21, 2012.

(51) Int. Cl. C12N 15/82

(52) U.S. Cl. CPC C12N 15/8273
USPC 800/278; 536/24.1; 435/320.1; 435/419; 800/298

(57) ABSTRACT

The invention provides nucleotide sequences that can be used in operable association with a promoter to express a polynucleotide of interest in a plant, plant part or plant cell. Also provided are methods of increasing or decreasing the expression of a nucleotide sequence of interest in a plant, plant part or plant cell in response to nitrate, drought and/or rehydration.

### STATEMENT OF PRIORITY This application claims the benefit, under 35 U.S.C. §119 (e), of U.S. Provisional Application No. 61/649,757 was filed on May 21, 2012, the entire contents of which is incorporated by reference herein.

### STATEMENT REGARDING ELECTRONIC FILING OF A SEQUENCE LISTING

A Sequence Listing in ASCII text format, submitted under 37 C.F.R. §1.821, entitled 9207-77TS_ST25.txt, 4526 bytes in size, generated on May 10, 2013 and filed via EFS-Web, is provided in lieu of a paper copy. This Sequence Listing is hereby incorporated herein by reference into the specification for its disclosures.

### FIELD OF THE INVENTION

The present invention relates to methods of introducing and expressing nucleotide sequences in a plant, plant part or plant cell.

### BACKGROUND OF THE INVENTION

Plants are subject to various stress conditions that may adversely affect their productivity. For instance, plants are grown in the regions of the world that experience drought or intermittent rainfall, the latter causing cycles of water limitation followed by rehydration. Numerous studies have demonstrated how plants respond to drought and subsequent rehydration. Conserved responses to drought at the whole plant or organ level include changes in the cell wall to reduce water loss, altered root architecture, reduced stomatal conductance, and induction of macromolecule osmoprotectants including Late Embryogenesis Abundant (LEA) proteins, chaperones, proline, alcohol sugars and trehalose, as well as various osmotic solutes (Na⁺, K⁺, Ca²⁺, Cl⁻) which act to balance the cellular osmotic pressure.

The hormone abscisic acid (ABA) coordinates many responses to drought. ABA-drought signalling involves a signal transduction cascade involving phospholipids, calcium-dependent kinases and G proteins. Downstream responses are mediated by various transcription factor families including dehydration response element binding protein/C-repeat binding factors (DREB/CBF), which are members of the ERF/AP2-type transcription factor family and REB/CBF proteins target genes containing DRE/CRT cis-acting promoter motifs. Additional transcription factors involved in drought signalling include MYC/MYB, NAC, WRKY and bZIP families. In addition to ABA, other hormones implicated in drought and/or rehydration responses include jasmonates in particular, in addition to ethylene, auxin, and gibberellins (GA).

Productivity and growth can also be affected by soil nitrogen. Maize (Zea mays L., corn) is one of the world's three most important food crops but its growth is limited by soil nitrogen. Nitrogen fertilizer is expensive and a leading cause of reduced farm income and food insecurity worldwide. It is estimated that only about 50% of nitrogen fertilizers are taken up by maize roots, with the remainder leached into groundwater or volatilized, contributing to environmental degradation.

Nitrate (NO₃⁻) is a critical inorganic form of nitrogen nutrition for many plant species including maize. Plants have evolved different nitrogen-uptake transporter systems to cope with the wide variation in soil nitrate concentrations. At high nitrate, there is a low-affinity transport system encoded by the Nrt1 gene family, whereas at low nitrate, there is a high-affinity transport system encoded by the Nrt2 and Nar2 gene families. In tomato, expression of Nrt1.1 was restricted to RH after nitrate exposure, while Nrt1.2 was also expressed in Arabidopsis RH and tomato RH though not specifically. These observations demonstrate the importance of RH for nitrate uptake. In general, expression of genes encoding nitrogen transporters and assimilation genes in RH has been understudied in maize.

Following nitrate uptake by epidermal cells, it is assimilated into amino acids for export to the rest of the plant, requiring coordination of nitrogen, carbon and other mineral nutrition pathways. Several regulatory molecules have been implicated in this coordination including the transcription factor Dof1. Nitrogen uptake and assimilation are energetically expensive processes, requiring further coordination with sugar breakdown and energy metabolism pathways. In mammals, it has been shown that when nutrient availability is low, general protein translation is also modulated, a process mediated in part by the Target of Rapamycin (TOR) signalling pathway.

### SUMMARY OF THE INVENTION

The present invention provides methods and compositions for expressing polynucleotides of interest in plants, plant parts and plant cells in response to environmental stimuli including nitrogen levels (e.g., nitrate) and/or drought and/or rehydration.

Accordingly, in one aspect, the invention provides an isolated nucleic acid comprising a promoter having one or more nucleotide sequences of SEQ ID NO:1, SEQ ID NO:2, SEQ ID NO:3, SEQ ID NO:4, SEQ ID NO:5, SEQ ID NO:6, SEQ ID NO:7, and/or SEQ ID NO:8, wherein the promoter modulates transcription of an operably linked polynucleotide in response to nitrate (NO₃).

A further aspect of the present invention provides an isolated nucleic acid comprising a promoter having one or more nucleotide sequences of SEQ ID NO:9, SEQ ID NO:10, SEQ ID SEQ ID NO:12, SEQ ID NO:13, SEQ ID NO:14, SEQ ID NO:15, SEQ ID NO:16, SEQ ID NO:17, SEQ ID NO:18, SEQ ID NO:19, and/or SEQ ID NO:20, wherein the promoter modulates transcription of an operably linked polynucleotide in response to drought and/or rehydration.

In some aspects of the invention, a promoter comprising one or more nucleotide sequences of this invention (e.g., SEQ ID NO:1, SEQ ID NO:2, SEQ ID NO:3, SEQ ID NO:4, SEQ ID NO:5, SEQ ID NO:6, SEQ ID NO:7, and SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, and/or SEQ ID NO:20) is operably linked to a polynucleotide sequence of interest. In some aspects, the promoter comprising nucleotide sequences of this invention can be a minimal promoter. In other aspects of the invention, the promoter comprising nucleotide sequences of this invention can direct leaf-specific transcription or root-preferred transcription.

The present invention further provides an expression cassette and/or a vector comprising a nucleic acid of this invention. Additionally, the present invention provides plants, plant parts and/or cells and/or progeny thereof comprising a nucleic acid, an expression cassette, and/or a vector of the present invention.

A further aspect of the invention provides a method of modulating the expression a polynucleotide of interest in a plant in response to nitrate (NO₃), the method comprising: introducing into a plant cell a nucleic acid of the invention, an expression cassette of the invention and/or a vector of the invention to produce a transformed plant cell; regenerating a transformed plant from the transformed plant cell; and exposing the transformed plant, or a plant part, or plant cell therefrom, to NO₃. In some aspects of the invention, the nucleic acid, the expression cassette, and/or the vector comprise a promoter, wherein the promoter is operably linked to one or more nucleotide sequences of SEQ ID NO:1, SEQ ID NO:2, SEQ ID NO:3, or any combination thereof, and the expression of the polynucleotide is increased in response to nitrate. In other aspects of the invention, the nucleic acid, the expression cassette, and/or the vector comprise a promoter, wherein the promoter is operably linked to one or more nucleotide sequences of SEQ ID NO:4, SEQ ID NO:5, SEQ ID NO:6, SEQ ID NO:7, SEQ ID NO:8, or any combination thereof, and the expression of the polynucleotide is decreased in response to nitrate.

In another aspect of the invention, a method of modulating the expression of a polynucleotide of interest in a plant in response to drought is provided, the method comprising: introducing into a plant cell a nucleic acid of the invention, an expression cassette of the invention and/or a vector of the invention to produce a transformed plant cell; regenerating a transformed plant from the transformed plant cell; and exposing the transformed plant, or a plant part or plant cell therefrom, to drought. In some aspects of the invention, the nucleic acid, the expression cassette, and/or the vector comprise a promoter, wherein the promoter is operably linked to one or more nucleotide sequences of SEQ ID NO:9, SEQ ID NO:10, SEQ ID NO:19, SEQ ID NO:20, or any combination thereof, and the expression of the polynucleotide is increased in response to drought. In other aspects of the invention, the nucleic acid, the expression cassette, and/or the vector comprise a promoter, wherein the promoter is operably linked to one or more nucleotide sequences of SEQ ID NO:1 1, SEQ ID NO:12, SEQ ID NO:13, SEQ ID NO:14, SEQ ID NO:15, SEQ ID NO:16, SEQ ID NO:17, SEQ ID NO:18, or any combination thereof, and the expression of the polynucleotide is decreased in response to drought.

A further aspect of the invention provides a method of modulating the expression of a polynucleotide of interest in a plant in response to rehydration, the method comprising: introducing into a plant cell a nucleic acid of the invention, an expression cassette of the invention and/or a vector of the invention to produce a transformed plant cell; regenerating a transformed plant from the transformed plant cell; and rehydrating the transformed plant, or a plant part or plant cell therefrom. In some aspects of the invention, the nucleic acid, the expression cassette, and/or the vector comprise a promoter, wherein the promoter is operably linked to one or more nucleotide sequences of SEQ ID NO:9, SEQ ID NO:10, SEQ ID NO:19, SEQ ID NO:20, or any combination thereof, and the expression of the polynucleotide is decreased in response to rehydration. In other aspects of the invention, the nucleic acid, the expression cassette, and/or the vector comprise a promoter, wherein the promoter is operably linked to one or more nucleotide sequences of SEQ ID NO:11, SEQ ID NO:12, SEQ ID NO:13, SEQ ID NO:14, SEQ ID NO:15, SEQ ID NO:16, SEQ ID NO:17, SEQ ID NO:18, or any combination thereof, and the expression of the polynucleotide is increased in response to rehydration.

A further aspect of the invention provides a method of producing a plant comprising a nucleic acid of this invention, an expression cassette of this invention and/or a vector of this invention, the method comprising: introducing into a plant cell a nucleic acid of this invention, an expression cassette of this invention and/or a vector of this invention to produce a stably transformed plant cell; and regenerating a stably transformed plant from the plant cell.

The present invention further provides transgenic plants, plants parts including seeds comprising the nucleic acids of this invention, crops comprising said plants, and products produced from the transgenic plants and plant parts of this invention.

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

### DETAILED DESCRIPTION OF THE INVENTION

It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

Unless the context indicates otherwise, it is specifically intended that the various features of the invention described herein can be used in any combination.

Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

The present invention provides compositions and methods for expressing nucleotide sequences in a plant, plant part or plant cell in response to environmental factors such as nitrogen and water availability. Specifically, the present inventors have used microarray analysis to identify gene expression clusters in roots and root hairs. These gene expression clusters were then used to identify over-represented motifs in the promoters of the genes within expression clusters. The identified motifs described herein may be used to effect transcription of polynucleotide sequences of interest in response to nitrate, drought and rehydration.

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The term “about,” as used herein when referring to a measurable value such as a dosage or time period and the like, is meant to encompass variations of ±20%, ±10%, ±5%, ±1%, ±0.5%, or even ±0.1% of the specified amount.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

Unless indicated otherwise, the term “drought” refers to any condition whereby a plant is under water stress (e.g., lack of rain, or lack of watering; soil or media conditions affecting water availability).

Unless indicated otherwise, the term “rehydration” refers to exposure of a plant to sufficient water that was previously under drought conditions.

Unless indicated otherwise, the phrase “exposing to nitrate” refers to contacting the plant or part thereof (e.g., root and/or root hair) with nitrate.

The term “modulate” (and grammatical variations) refers to an increase or decrease.

As used herein, the terms “increase,” “increases,” “increased,” “increasing” and similar terms indicate an elevation of at least about 5%, 10%, 15%, 20%, 25%, 30%, 35%, 40%, 45%, 50%, 55%, 60%, 75%, 80%,85%, 90%, 95%, 100%, 150%, 200%, 300%, 400%, 500% or more as compared to a control (e.g., a plant that does not comprise at least one isolated nucleic acid of the present invention).

As used herein, the terms “reduce,” “reduces,” “reduced,” “reduction” and similar terms mean a decrease of at least about 5%, 10%, 15%, 20%, 25%, 30%, 35%, 40%, 45%, 50%, 55%, 60%, 75%, 80%,85%, 90%, 95%, 96%, 97%, 98%, 99% or 100% as compared to a control (e.g., a plant that does not comprise at least one isolated nucleic acid of the present invention). In particular embodiments, the reduction results in no or essentially no (i.e., an insignificant amount, e.g., less than about 10%, less than about 5% or even less than about 1%) detectable activity or amount.

As used herein, the term “heterologous” means foreign, exogenous, non-native and/or non-naturally occurring.

As used here, “homologous” means native. For example, a homologous nucleotide sequence or amino acid sequence is a nucleotide sequence or amino acid sequence naturally associated with a host cell into which it is introduced, a homologous promoter sequence is the promoter sequence that is naturally associated with a coding sequence, and the like.

As used herein a “chimeric nucleic acid,” “chimeric nucleotide sequence” or “chimeric polynucleotide” comprises a promoter that is operably linked to one or more nucleotide sequence of this invention (e.g., SEQ ID NOs:1-20) and/or to a polynucleotide of interest each of which are heterologous to the promoter (or vice versa). In particular embodiments, the “chimeric nucleic acid,” “chimeric nucleotide sequence” or “chimeric polynucleotide” comprises a nucleic acid encoding a promoter sequence that is operably linked to one or more nucleotide sequences of this invention and to a heterologous polynucleotide of interest.

A “promoter” is a nucleotide sequence that controls or regulates the transcription of a nucleotide sequence (i.e., a coding sequence) that is operatively associated with the promoter. The coding sequence may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. The promoter region may comprise other elements that act as regulators of gene expression. These include a TATA box consensus sequence, and often a CAAT box consensus sequence (Breathnach and Chambon, (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227).

The nucleotide sequences of the present invention (e.g., SEQ ID NOs:1-20) can be used in combination with any heterologous promoter nucleotide sequence (e.g., the one or more nucleotide sequences of this invention can be operably associated with a promoter), thereby producing a recombinant or synthetic promoter that is responsive to nitrate, drought and/or rehydration. Thus, in some embodiments, the present invention excludes promoters that are homologous (native) to the nucleotide sequences of the present invention (e.g., SEQ ID NOs:1-20).

A “heterologous promoter” is any promoter that is heterologous (e.g., foreign or non-native) to the nucleotide sequence of the invention (e.g., a promoter motif as described herein) with which it is operably associated.

The choice of promoters useable with the present invention can be made among many different types of promoters. Thus, the choice of promoter depends upon several factors, including, but not limited to, cell- or tissue-specific/tissue-preferential expression, desired expression level, efficiency, inducibility and/or selectability. For example, where expression in a specific tissue or organ is desired in addition to inducibility, a tissue-specific promoter can be used (e.g., a root specific promoter). In contrast, where expression in response to a stimulus is desired a promoter inducible by other stimuli or chemicals can be used. Where continuous expression at a relatively constant level is desired throughout the cells of a plant a constitutive promoter can be chosen.

Non-limiting examples of constitutive promoters include cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter.

Some non-limiting examples of tissue-specific/tissue-preferential promoters useable with the present invention include those encoding the seed storage proteins (such as β-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP Patent No. 255378). Thus, in some embodiments, the promoters associated with these tissue-specific nucleic acids can be used in the present invention.

Additional examples of tissue-specific/tissue-preferential promoters include, but are not limited to, the promoters comprising root hair—specific cis-elements (RHEs) (Kim et al. The Plant Cell 18:2958-2970 (2006)), root-specific promoters RCc3 (Jeong et al. Plant Physiol. 153:185-197 (2010)) and RB7 (U.S. Pat. No. 5459252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology, 37(8):1108-1115), corn light harvesting complex promoter (Bansal et al. (1992) Proc. Natl. Acad. Sci. USA 89:3654-3658), corn heat shock protein promoter (O'Dell et al. (1985) EMBO J. 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” 29-39 In: Genetic Engineering of Plants (Hollaender ed., Plenum Press 1983; and Poulsen et al. (1986) Mol. Gen. Genet. 205:193-200), Ti plasmid mannopine synthase promoter (Langridge et al. (1989) Proc. Natl. Acad. Sci, USA 86:3219-3223), Ti plasmid nopaline synthase promoter (Langridge et al. (1989), supra), petunia chalcone isomerase promoter (van Tunen et al. (1988) EMBO 1 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612). Particularly useful for seed-specific expression is the pea vicilin promoter (Czako et al. (1992) Mol. Gen. Genet. 235:33-40; as well as U.S. Pat. No. 5,625,136). Other useful promoters for expression in mature leaves are those that are switched on at the onset of senescence, such as the SAG promoter from Arabidopsis (Gan et al. (1995) Science 270:1986-1988).

In addition, promoters functional in plastids can be used. Non-limiting examples of such promoters include the bacteriophage T3 gene 9 5′ UTR and other promoters disclosed in U.S. Pat. No. 7,579,516. In other embodiments, promoters useful with the present invention include but are not limited to the S-E9 small subunit RuBP carboxylase promoter and the Kunitz trypsin inhibitor gene promoter (Kti3).

In some instances, inducible promoters are useable with the present invention. Examples of inducible promoters useable with the present invention include, but are not limited to, tetracycline repressor system promoters, Lac repressor system promoters, copper-inducible system promoters, salicylate-inducible system promoters (e.g., the PR1a system), glucocorticoid-inducible promoters (Aoyama et al. (1997) Plant J. 11:605-612), and ecdysone-inducible system promoters. Other non-limiting examples of inducible promoters include ABA- and turgor-inducible promoters, the auxin-binding protein gene promoter (Schwob et al. (1993) Plant J. 4:423-432), the UDP glucose flavonoid glycosyl-transferase promoter (Ralston et al. (1988) Genetics 119:185-197), the MPI proteinase inhibitor promoter (Cordero et al. (1994) Plant 1 6:141-150), the glyceraldehyde-3-phosphate dehydrogenase promoter (Kohler et al. (1995) Plant Mol. Biol. 29:1293-1298; Martinez et al. (1989) J. Mol. Biol. 208:551-565; and Quigley et al. (1989) J. Mol. Evol. 29:412-421) the benzene sulphonamide-inducible promoters (U.S. Pat. No. 5,364,780) and the glutathione S-transferase promoters. Likewise, one can use any appropriate inducible promoter described in Gatz (1996) Current Opinion Biotechnol. 7:168-172 and Gatz (1997) Annu. Rev. Plant Physiol. Plant Mol. Biol. 48:89-108.

In some embodiments of the present invention, a “minimal promoter” or “basal promoter” is used. A minimal promoter is capable of recruiting and binding RNA polymerase II complex and its accessory proteins to permit transcriptional initiation and elongation. In some embodiments, a minimal promoter is constructed to comprise only the nucleotides/nucleotide sequences from a selected promoter that are required for binding of the transcription factors and transcription of a nucleotide sequence of interest that is operably associated with the minimal promoter including but not limited to TATA box sequences. In other embodiments, the minimal promoter lacks cis sequences that recruit and bind transcription factors that modulate (e.g., enhance, repress, confer tissue specificity, confer inducibility or repressibility) transcription. A minimal promoter is generally placed upstream (i.e., 5′) of a nucleotide sequence to be expressed. Thus, nucleotides/nucleotide sequences from any promoter useable with the present invention can be selected for use as a minimal promoter.

Any promoter, such as those described herein, may be altered to generate a minimal promoter by progressively removing nucleotides from the promoter until the promoter ceases to function in order to identify the minimal promoter. Thus, the smallest fragment of a promoter which still functions as a promoter can also be considered a minimal promoter. Accordingly, in some embodiments, a minimal promoter comprising the nucleotide sequences of the invention can be used to drive developmental gene expression in plants. In some particular embodiments, the minimal promoter is a CaMV 35S minimal promoter. Thus, in some embodiments of the invention, a minimal promoter can be operably linked to one or more nucleotide sequences of the invention (e.g., SEQ ID NOs:1-20) and thereby conferring developmental stage- or tissue-specific/tissue-preferential transcription upon a polynucleotide sequence of interest that is also operably linked to said promoter.

Thus, any promoter suitable for use with this invention can be manipulated to produce synthetic or chimeric promoters that combine cis elements from two or more promoters, for example, by adding a heterologous regulatory sequence to an active promoter with its own partial or complete regulatory sequences (Ellis et al., EMBO J. 6:11 16, 1987; Strittmatter and Chua, Proc. Nat. Acad. Sci. USA 84:8986 8990, 1987; Poulsen and Chua, Mol. Gen. Genet. 214:16 23, 1988; Comai et al., Plant. Mol. Biol. 15:373 381, 1991) (See also U.S. Pat. No. 7,202,085). Alternatively, a synthetic promoter can be produced by adding one or more heterologous regulatory sequences (e.g., the nucleotide sequences of this invention, SEQ ID NOs:1-20) to the 5′ upstream region of minimal promoter, (Fluhr et al., Science 232:1106 1112, 1986; Strittmatter and Chua, Proc. Nat. Acad. Sci. USA 84:8986 8990, 1987; Aryan et al., Mol. Gen. Genet. 225:65 71, 1991; Shen and Ho, Physiol. Plantarum 101:653-664 (1997)). Cis elements such as the nucleotide sequences of this invention (SEQ ID NOs:1-20) can be obtained by chemical synthesis or by cloning from promoters that includes such elements, and they can be synthesized with additional flanking sequences that contain useful restriction enzyme sites to facilitate subsequent manipulation.

“Polynucleotide of interest” or “nucleotide sequence of interest” or refers to any polynucleotide sequence which, when introduced into a plant, confers upon the plant a desired characteristic such as tolerance to abiotic stress, antibiotic resistance, virus resistance, insect resistance, disease resistance, or resistance to other pests, herbicide tolerance, improved nutritional value, improved performance in an industrial process or altered reproductive capability. The “polynucleotide of interest” may also be one that is transferred to plants for the production of commercially valuable products such as enzymes or metabolites in the plant. The “polynucleotide of interest” can encode a polypeptide and/or an inhibitory polynucleotide (e.g., a functional RNA).

A “heterologous polynucleotide of interest” is heterologous (e.g., foreign) to the promoter with which it is operatively associated. Thus, a polynucleotide sequence of interest that is operatively associated with a recombinant or synthetic promoter comprising the polynucleotide sequences of the invention (e.g., SEQ ID NOs:1-20) as described herein is heterologous to that recombinant/synthetic promoter.

A “functional” RNA includes any untranslated RNA that has a biological function in a cell, e.g., regulation of gene expression. Such functional RNAs include but are not limited to RNAi (e.g., siRNA, shRNA), miRNA, antisense RNA, ribozymes, RNA aptamers and the like.

By “operably linked” or “operably associated” as used herein, it is meant that the indicated elements are functionally related to each other, and are also generally physically related. For example, a promoter is operatively linked or operably associated to a coding sequence (e.g., polynucleotide of interest) if it controls the transcription of the sequence. Thus, the term “operatively linked” or “operably associated” as used herein, refers to nucleotide sequences on a single nucleic acid molecule that are functionally associated. Those skilled in the art will appreciate that the control sequences (e.g., promoter) need not be contiguous with the coding sequence, as long as they functions to direct the expression thereof. Thus, for example, intervening untranslated, yet transcribed, sequences can be present between a promoter and a coding sequence, and the promoter sequence can still be considered “operably linked” to the coding sequence.

By the term “express,” “expressing” or “expression” (or other grammatical variants) of a nucleic acid coding sequence, it is meant that the sequence is transcribed. In particular embodiments, the terms “express,” “expressing” or “expression” (or other grammatical variants) can refer to both transcription and translation to produce an encoded polypeptide.

The term “abiotic stress” as used herein refers to outside, nonliving, factors which can cause harmful effects to plants. Thus, as used herein, abiotic stress includes, but is not limited to, drought, and/or rehydration, and/or combinations thereof. Parameters for the abiotic stress factors are species specific and even variety specific and therefore vary widely according to the species/variety exposed to the abiotic stress. In addition, because most crops are exposed to multiple abiotic stresses at one time, the interaction between the stresses affects the response of the plant. Thus, the particular parameters for high/low temperature, light intensity, drought and the like, which impact crop productivity, will vary with species, variety, degree of acclimatization and the exposure to a combination of environmental conditions.

“Wild-type” nucleotide sequence or amino acid sequence refers to a naturally occurring (“native”) or endogenous nucleotide sequence (including a cDNA corresponding thereto) or amino acid sequence.

The terms “nucleic acid,” “polynucleotide” and “nucleotide sequence” are used interchangeably herein unless the context indicates otherwise. These terms encompass both RNA and DNA, including cDNA, genomic DNA, partially or completely synthetic (e.g., chemically synthesized) RNA and DNA, and chimeras of RNA and DNA. The nucleic acid, polynucleotide or nucleotide sequence may be double-stranded or single-stranded, and further may be synthesized using nucleotide analogs or derivatives (e.g., inosine or phosphorothioate nucleotides). Such nucleotides can be used, for example, to prepare nucleic acids, polynucleotides and nucleotide sequences that have altered base-pairing abilities or increased resistance to nucleases. The present invention further provides a nucleic acid, polynucleotide or nucleotide sequence that is the complement (which can be either a full complement or a partial complement) of a nucleic acid, polynucleotide or nucleotide sequence of the invention. Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 CFR §1.822 and established usage.

The nucleic acids and polynucleotides of the invention can be isolated. An “isolated” nucleic acid molecule or polynucleotide is a nucleic acid molecule or polynucleotide that, by the hand of man, exists apart from its native environment and is therefore not a product of nature. An isolated nucleic acid molecule or isolated polynucleotide may exist in a purified form or may exist in a non-native environment such as, for example, a recombinant host cell. Thus, for example, the term “isolated” means that it is separated from the chromosome and/or cell in which it naturally occurs. A nucleic acid or polynucleotide is also isolated if it is separated from the chromosome and/or cell in which it naturally occurs and is then inserted into a genetic context, a chromosome, a chromosome location, and/or a cell in which it does not naturally occur. The recombinant nucleic acid molecules and polynucleotides of the invention can be considered to be “isolated.”

Further, an “isolated” nucleic acid or polynucleotide can be a nucleotide sequence (e.g., DNA or RNA) that is not immediately contiguous with nucleotide sequences with which it is immediately contiguous (one on the 5′ end and one on the 3′ end) in the naturally occurring genome of the organism from which it is derived. The “isolated” nucleic acid or polynucleotide can exist in a cell (e.g., a plant cell), optionally stably incorporated into the genome. According to this embodiment, the “isolated” nucleic acid or polynucleotide can be foreign to the cell/organism into which it is introduced, or it can be native to an the cell/organism, but exist in a recombinant form (e.g., as a chimeric nucleic acid or polynucleotide) and/or can be an additional copy of an endogenous nucleic acid or polynucleotide. Thus, an “isolated nucleic acid molecule” or “isolated polynucleotide” can also include a nucleotide sequence derived from and inserted into the same natural, original cell type, but which is present in a non-natural state, e.g., present in a different copy number, in a different genetic context and/or under the control of different regulatory sequences than that found in the native state of the nucleic acid molecule or polynucleotide.

In representative embodiments, the “isolated” nucleic acid or polynucleotide is substantially free of cellular material (including naturally associated proteins such as histones, transcription factors, and the like), viral material, and/or culture medium (when produced by recombinant DNA techniques), or chemical precursors or other chemicals (when chemically synthesized). Optionally, in representative embodiments, the isolated nucleic acid or polynucleotide is at least about 10%, 20%, 30%, 40%, 50%, 60%, 70%, 80%, 90%, 95% or more pure.

As used herein, the term “recombinant” nucleic acid, polynucleotide or nucleotide sequence refers to a nucleic acid, polynucleotide or nucleotide sequence that has been constructed, altered, rearranged and/or modified by genetic engineering techniques. The term “recombinant” does not refer to alterations that result from naturally occurring events, such as spontaneous mutations, or from non-spontaneous mutagenesis.

A “vector” is any nucleic acid molecule for the cloning of and/or transfer of a nucleic acid into a cell. A vector may be a replicon to which another nucleotide sequence may be attached to allow for replication of the attached nucleotide sequence. A “replicon” can be any genetic element (e.g., plasmid, phage, cosmid, chromosome, viral genome) that functions as an autonomous unit of nucleic acid replication in the cell, i.e., capable of nucleic acid replication under its own control. The term “vector” includes both viral and nonviral (e.g., plasmid) nucleic acid molecules for introducing a nucleic acid into a cell in vitro, ex vivo, and/or in vivo, and is optionally an expression vector. A large number of vectors known in the art may be used to manipulate, deliver and express polynucleotides. Vectors may be engineered to contain sequences encoding selectable markers that provide for the selection of cells that contain the vector and/or have integrated some or all of the nucleic acid of the vector into the cellular genome. Such markers allow identification and/or selection of host cells that incorporate and express the proteins encoded by the marker. A “recombinant” vector refers to a viral or non-viral vector that comprises one or more nucleotide sequences of interest (e.g., transgenes), e.g., two, three, four, five or more polynucleotide sequences of interest.

Viral vectors have been used in a wide variety of gene delivery applications in cells, as well as living animal subjects. Plant viral vectors that can be used include, but are not limited to, geminivirus vectors and/or tobomovirus vectors. Non-viral vectors include, but are not limited to, plasmids, liposomes, electrically charged lipids (cytofectins), nucleic acid-protein complexes, and biopolymers. In addition to a nucleic acid of interest, a vector may also comprise one or more regulatory regions, and/or selectable markers useful in selecting, measuring, and monitoring nucleic acid transfer results (e.g., delivery to specific tissues, duration of expression, etc.).

The term “fragment,” as applied to a nucleic acid or polynucleotide, will be understood to mean a nucleotide sequence of reduced length relative to the reference or full-length nucleotide sequence and comprising, consisting essentially of, or consisting of contiguous nucleotides from the reference or full-length nucleotide sequence. Such a fragment according to the invention may be, where appropriate, included in a larger polynucleotide of which it is a constituent. In some embodiments, such fragments can comprise, consist essentially of and/or consist of oligonucleotides having a length that greater than and/or is at least about 8, 10, 12, 15, 16, 17, 18, 19, 20, 25, 30, 35, 40, 45, 50, 75, 100, 125, 150, 175, 200, 250, 300, 350, 400, 450, 500, 600, 700, 800, 900, 1000 nucleotides (optionally, contiguous nucleotides) or more from the reference or full-length nucleotide sequence, as long as the fragment is shorter than the reference or full-length nucleotide sequence. In representative embodiments, the fragment is a biologically active nucleotide sequence, as that term is described herein.

A “biologically active” nucleotide sequence is one that substantially retains at least one biological activity normally associated with the wild-type nucleotide sequence, for example, promoter activity, optionally inducible promoter activity in response to exposure to nitrate, drought or rehydration. In particular embodiments, the “biologically active” nucleotide sequence substantially retains all of the biological activities possessed by the umnodified sequence. By “substantially retains” biological activity, it is meant that the nucleotide sequence retains at least about 50%, 60%, 75%, 85%, 90%, 95%, 97%, 98%, 99%, or more, of the biological activity of the native nucleotide sequence (and can even have a higher level of activity than the native nucleotide sequence). Methods of measuring promoter activity are known in the art.

Two nucleotide sequences are said to be “substantially identical” to each other when they share at least about 60%, 70%, 75%, 80%, 85%, 90%, 95%, 97%, 98%, 99% or even 100% sequence identity. In some particular embodiments, the nucleotide sequences of the present invention include nucleotides sequences having 90%, 95%, 97%, 98%, or 99% sequence identity to the nucleotide sequences of the invention (e.g., SEQ ID NOs:1-20)

Two amino acid sequences are said to be “substantially identical” or “substantially similar” to each other when they share at least about 60%, 70%, 75%, 80%, 85%, 90%, 95%, 97%, 98%, 99% or even 100% sequence identity or similarity, respectively.

As used herein “sequence identity” refers to the extent to which two optimally aligned polynucleotide or polypeptide sequences are invariant throughout a window of alignment of components, e.g., nucleotides or amino acids.

As used herein “sequence similarity” is similar to sequence identity (as described herein), but permits the substitution of conserved amino acids (e.g., amino acids whose side chains have similar structural and/or biochemical properties), which are well-known in the art.

As is known in the art, a number of different programs can be used to identify whether a nucleic acid has sequence identity or an amino acid sequence has sequence identity or similarity to a known sequence. Sequence identity or similarity may be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J. Mol. Biol. 48,443 (1970), by the search for similarity method of Pearson & Lipman, Proc. Natl. Acad. Sci. USA 85,2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12, 387-395 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35, 351-360 (1987); the method is similar to that described by Higgins & Sharp, CABIOS 5, 151-153 (1989).

Another example of a useful algorithm is the BLAST algorithm, described in Altschul et al., J. Mol. Biol. 215, 403-410, (1990) and Karlin et al., Proc. Natl. Acad Sci. USA 90, 5873-5787 (1993). A particularly useful BLAST program is the WU-BLAST-2 program which was obtained from Altschul et al., Methods in Enzymology, 266, 460-480 (1996); http://blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al. Nucleic Acids Res. 25, 3389-3402 (1997).

The CLUSTAL program can also be used to determine sequence similarity. This algorithm is described by Higgins et al. (1988) Gene 73:237; Higgins et al. (1989) CABIOS 5:151-153; Corpet et al. (1988) Nucleic Acids Res. 16: 10881-90; Huang et al. (1992) CABIOS 8: 155-65; and Pearson et al. (1994) Meth. Mol. Biol. 24: 307-331.

The alignment may include the introduction of gaps in the sequences to be aligned. In addition, for sequences which contain either more or fewer nucleotides than the nucleic acids disclosed herein, it is understood that in one embodiment, the percentage of sequence identity will be determined based on the number of identical nucleotides acids in relation to the total number of nucleotide bases. Thus, for example, sequence identity of sequences shorter than a sequence specifically disclosed herein, will be determined using the number of nucleotide bases in the shorter sequence, in one embodiment. In percent identity calculations relative weight is not assigned to various manifestations of sequence variation, such as, insertions, deletions, substitutions, etc.

Two nucleotide sequences can also be considered to be substantially identical when the two sequences hybridize to each other under stringent conditions. A nonlimiting example of “stringent” hybridization conditions include conditions represented by a wash stringency of 50% formamide with 5× Denhardt's solution, 0.5% SDS and 1×SSPE at 42° C. “Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). In some representative embodiments, two nucleotide sequences considered to be substantially identical hybridize to each other under highly stringent conditions. Generally, highly stringent hybridization and wash conditions are selected to be about 5° C. lower than the thermal melting point (Tₘ) for the specific sequence at a defined ionic strength and pH.

As used herein, the term “polypeptide” encompasses both peptides and proteins (including fusion proteins), unless indicated otherwise.

A “fusion protein” is a polypeptide produced when two heterologous nucleotide sequences or fragments thereof coding for two (or more) different polypeptides not found fused together in nature are fused together in the correct translational reading frame.

An “isolated” polypeptide is a polypeptide that, by the hand of man, exists apart from its native environment and is therefore not a product of nature. An isolated polypeptide may exist in a purified form or may exist in a non-native environment such as, for example, a recombinant host cell.

In representative embodiments, an “isolated” polypeptide means a polypeptide that is separated or substantially free from at least some of the other components of the naturally occurring organism or virus, for example, the cell or viral structural components or other polypeptides or nucleic acids commonly found associated with the polypeptide. In particular embodiments, the “isolated” polypeptide is at least about 1%, 5%, 10%, 25%, 50%, 60%, 70%, 75%, 80%, 85%, 90%, 95%, 97%, 98%, 99% or more pure (w/w). In other embodiments, an “isolated” polypeptide indicates that at least about a 5-fold, 10-fold, 25-fold, 100-fold, 1000-fold, 10,000-fold, or more enrichment of the protein (w/w) is achieved as compared with the starting material.

A “biologically active” polypeptide is one that substantially retains at least one biological activity normally associated with the wild-type polypeptide. In particular embodiments, the “biologically active” polypeptide substantially retains all of the biological activities possessed by the unmodified (e.g., native) sequence. By “substantially retains” biological activity, it is meant that the polypeptide retains at least about 50%, 60%, 75%, 85%, 90%, 95%, 97%, 98%, 99%, or more, of the biological activity of the native polypeptide (and can even have a higher level of activity than the native polypeptide).

“Introducing” in the context of a plant cell, plant tissue, plant part and/or plant means contacting a nucleic acid molecule with the plant cell, plant tissue, plant part, and/or plant in such a manner that the nucleic acid molecule gains access to the interior of the plant cell or a cell of the plant tissue, plant part or plant. Where more than one nucleic acid molecule is to be introduced, these nucleic acid molecules can be assembled as part of a single polynucleotide or nucleic acid construct, or as separate polynucleotide or nucleic acid constructs, and can be located on the same or different nucleic acid constructs. Accordingly, these polynucleotides can be introduced into plant cells in a single transformation event, in separate transformation events, or, e.g., as part of a breeding protocol.

The term “transformation” as used herein refers to the introduction of a heterologous and/or isolated nucleic acid into a cell. Transformation of a cell may be stable or transient. Thus, a transgenic plant cell, plant tissue, plant part and/or plant of the invention can be stably transformed or transiently transformed.

“Transient transformation” in the context of a polynucleotide means that a polynucleotide is introduced into the cell and does not integrate into the genome of the cell.

As used herein, “stably introducing,” “stably introduced,” “stable transformation” or “stably transformed” (and similar terms) in the context of a polynucleotide introduced into a cell, means that the introduced polynucleotide is stably integrated into the genome of the cell (e.g., into a chromosome or as a stable-extra-chromosomal element). As such, the integrated polynucleotide is capable of being inherited by progeny cells and plants.

“Genome” as used herein includes the nuclear and/or plastid genome, and therefore includes integration of a polynucleotide into, for example, the chloroplast genome. Stable transformation as used herein can also refer to a polynucleotide that is maintained extrachromosomally, for example, as a minichromosome.

As used herein, the terms “transformed” and “transgenic” refer to any plant, plant cell, plant tissue (including callus), or plant part that contains all or part of at least one recombinant or isolated nucleic acid, polynucleotide or nucleotide sequence. In representative embodiments, the recombinant or isolated nucleic acid, polynucleotide or nucleotide sequence is stably integrated into the genome of the plant (e.g., into a chromosome or as a stable extra-chromosomal element), so that it is passed on to subsequent generations of the cell or plant.

The term “plant part,” as used herein, includes but is not limited to reproductive tissues (e.g., petals, sepals, stamens, pistils, receptacles, anthers, pollen, flowers, fruits, flower bud, ovules, seeds, embryos, nuts, kernels, ears, cobs and husks); vegetative tissues (e.g., petioles, stems, roots, root hairs, root tips, pith, coleoptiles, stalks, shoots, branches, bark, apical meristem, axillary bud, cotyledon, hypocotyls, and leaves); vascular tissues (e.g., phloem and xylem); specialized cells such as epidermal cells, parenchyma cells, chollenchyma cells, schlerenchyma cells, stomates, guard cells, cuticle, mesophyll cells; callus tissue; and cuttings. The term “plant part” also includes plant cells, including plant cells that are intact in plants and/or parts of plants, plant protoplasts, plant tissues, plant organs plant cell tissue cultures, plant calli, plant clumps, and the like. As used herein, “shoot” refers to the above ground parts including the leaves and stems.

The term “tissue culture” encompasses cultures of tissue, cells, protoplasts and callus.

As used herein, “plant cell” refers to a structural and physiological unit of the plant, which typically comprise a cell wall but also includes protoplasts. A plant cell of the present invention can be in the form of an isolated single cell or can be a cultured cell or can be a part of a higher-organized unit such as, for example, a plant tissue (including callus) or a plant organ.

Any plant (or groupings of plants, for example, into a genus or higher order classification) can be employed in practicing the present invention including angiosperms and/or gymnosperms, monocots and/or dicots.

Exemplary plants include, but are not limited to corn (Zea mays), canola (Brassica napus, Brassica rapa ssp.), alfalfa (Medicago saliva), rice (Oryza sativa, including without limitation Indica and/or Japonica varieties), rape (Brassica napus), rye (Secale cereale), sorghum (Sorghum bicolor, Sorghum vulgare), sunflower (Helianthus annus), wheat (Trificum aestivum), soybean (Glycine max), tobacco (Nicotiana tobacum), potato (Solanum tuberosum), peanuts (Arachis hypogaea), cotton (Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), apple (Malus pumila), blackberry (Rubus), strawberry (Fragaria), walnut (Juglans regia), grape (Vitis vinifera), apricot (Prunus armeniaca), cherry (Prunus), peach (Prunus persica), plum (Prunus domestica), pear (Pyrus communis), watermelon (Citrullus vulgaris), duckweed (Lemna), oats (Avena sativa), barley (Hordium vulgare), vegetables, ornamentals, conifers, and turfgrasses (e.g., for ornamental, recreational or forage purposes), and biomass grasses (e.g., switchgrass and Miscanthus).

Vegetables include Solanaceous species (e.g., tomatoes; Lycopersicon esculentum), lettuce (e.g., Lactuea sativa), carrots (Caucus carota), cauliflower (Brassica oleracea), celery (Apium graveolens), eggplant (Solanum melongena), asparagus (Asparagus officinalis), ochra (Abelmoschus esculentus), green beans (Phaseolus vulgaris), lima beans (Phaseolus limensis), peas (Lathyrus spp.), members of the genus Cucurbita such as Hubbard squash (C. Hubbard), Butternut squash (C. moschata), Zucchini (C. pepo), Crookneck squash (C. crookneck), C. argyrosperma, C. argyrosperma ssp. sororia, C, digitata, C. ecuadorensis, C. foetidissima, C. lundelliana, and C. martinezii, and members of the genus Cucumis such as cucumber (Cucumis sativus), cantaloupe (C. cantalupensis), and musk melon (C. melo).

Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherima), and chrysanthemum.

Conifers, which may be employed in practicing the present invention, include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

Turfgrass include but are not limited to zoysia grass, bent grass, fescue grass, bluegrass, St. Augustine grass, Bermuda grass, buffalo grass, rye grass, and orchard grass.

Also included are plants that serve primarily as laboratory models, e.g., Arabidopsis.

### I. Promoter Motif Sequences.

The present invention provides nucleotide sequences (e.g., SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:l0, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, and/or SEQ ID NO:20) that can be operably associated with a heterologous promoter to produce a recombinant promoter that can confer responsiveness to nitrate exposure, drought and/or rehydration, thereby resulting in the expression of a polynucleotide of interest operably linked to said recombinant promoter in response to nitrate exposure, drought and/or rehydration.

Accordingly, in representative embodiments, the invention provides a nucleic acid (e.g., a recombinant or isolated nucleic acid) comprising, consisting essentially of, or consisting of a nucleotide sequence selected from the group consisting of: (a) SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, and SEQ ID NO:20; (c) a nucleotide sequence that hybridizes to the complete complement of the nucleotide sequence of (a) under stringent hybridization conditions; and (d) a nucleotide sequence having at least about 90%, 95%, 97%, 98%, 99% sequence identity to the nucleotide sequences of any of (a) to (b).

In some particular embodiments, the recombinant nucleic acid of the present invention does not comprise the nucleotide sequence of SEQ ID NO:19. In still other embodiments of the invention, the recombinant nucleic acid of the present invention does not comprise the nucleotide sequence of SEQ ID NO:3, SEQ ID NO:14, SEQ ID NO:15, and/or SEQ ID NO:16.

In some embodiments, the nucleotide sequence of the present invention is a biologically active promoter motif sequence that can confer responsiveness (i.e., modulate transcription of an operably linked polynucleotide of interest) to exposure to nitrate, drought and/or rehydration when comprised in a promoter that is operably associated with a polynucleotide sequence of interest to be expressed, wherein said promoter modulates the transcription of the operably linked polynucleotide in response to nitrate, drought and/or rehydration.

Thus, in exemplary embodiments, the present invention provides, an isolated nucleic acid comprising a recombinant promoter comprising, consisting essentially of, or consisting of one or more nucleotide sequences selected from the group consisting of SEQ ID NO:1, SEQ ID NO:2, SEQ ID NO:3, SEQ ID NO:4, SEQ ID NO:5, SEQ ID NO:6, SEQ ID NO:7, and SEQ ID NO:8, wherein the promoter directs transcription of an operably linked polynucleotide in response to nitrate (NO₃). In additional embodiments, the present invention provides an isolated nucleic acid comprising a recombinant promoter comprising, consisting essentially of, or consisting of one or more nucleotide sequences selected from the group consisting of SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, and SEQ ID NO:20, wherein the promoter modulates transcription of an operably linked polynucleotide in response to drought and/or rehydration. In representative embodiments, the one or more nucleotide sequences can be a combination of one or more different nucleotide sequences of the invention (e.g., SEQ ID NOs:1-20), one or more of the same nucleotide sequence of the present invention (e.g., SEQ ID NOs:1-20), or any combination thereof of the same or different nucleotide sequences of the invention.

In some aspects of the invention, a promoter comprising one or more nucleotide sequences of this invention is also operably linked to a polynucleotide sequence of interest. According to this embodiment, a recombinant promoter comprising a nucleotide sequence of the invention controls or regulates expression (e.g., transcription and, optionally, translation) of the polynucleotide of interest. The promoter comprising the nucleotide sequences of the invention can be any suitable promoter. In some embodiments, the promoter comprising the nucleotide sequences of the invention is a minimal promoter. In particular embodiments, the promoter can be a CaMV 35S minimal promoter. In some embodiments of the invention, the promoter can direct leaf-specific transcription or root-preferred transcription.

The present invention further provides an expression cassette and/or a vector comprising a nucleic acid of this invention. Additionally, the present invention provides transformed plants, plant parts and/or cells and/or progeny thereof comprising a nucleic acid, an expression cassette, and/or a vector of the present invention.

Thus, the invention also provides an expression cassette comprising, consisting essentially of, or consisting of a nucleic acid of the invention (e.g., a recombinant promoter comprising a nucleotide sequence of the invention (e.g., SEQ ID NOs:1-20), wherein the recombinant promoter is optionally in operable association with a polynucleotide of interest. The expression cassette can further have a plurality of restriction sites for insertion of a polynucleotide of interest to be operably linked to the regulatory regions. In particular embodiments, the expression cassette comprises more than one (e.g., two, three, four or more) nucleotide sequences of interest.

The invention further provides a vector comprising a nucleic acid of the invention (e.g., a recombinant promoter comprising, consisting essentially of, or consisting of a nucleotide sequence of the invention (e.g., SEQ ID NOs:1-20) or an expression cassette comprising a nucleic acid of the invention, wherein the recombinant promoter is optionally in operable association with a polynucleotide of interest.

The expression cassettes of the invention may further comprise a transcriptional termination sequence. Any suitable termination sequence known in the art may be used in accordance with the present invention. The termination region may be native with the transcriptional initiation region, may be native with the polynucleotide of interest, or may be derived from another source. Convenient termination regions are available from the Ti-plasmid of A. tumefaciens, such as the octopine synthetase and nopaline synthetase termination regions. See also, Guerineau et al., Mol. Gen, Genet. 262, 141 (1991); Proudfoot, Cell 64, 671 (1991); Sanfacon et al., Genes Dev. 5,141 (1991); Mogen et al., Plant Cell 2, 1261 (1990); Munroe et al., Gene 91, 151 (1990); Ballas et al., Nucleic Acids Res. 17, 7891 (1989); and Joshi et al., Nucleic Acids Res. 15, 9627 (1987). Additional exemplary termination sequences are the pea RubP carboxylase small subunit termination sequence and the Cauliflower Mosaic Virus 35S termination sequence. Other suitable termination sequences will be apparent to those skilled in the art.

Further, in particular embodiments, the polynucleotide sequence of interest can be operably associated with a translational start site. The translational start site can be the native translational start site associated with a heterologous polynucleotide of interest, or any other suitable translational start codon.

In illustrative embodiments, the expression cassette includes in the 5′ to 3′ direction of transcription, a promoter comprising, consisting essentially of, or consisting of a nucleotide sequence of the present invention (e.g., SEQ ID SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, and/or SEQ ID NO:20), a polynucleotide of interest, and a transcriptional and translational termination region functional in plants.

Those skilled in the art will understand that the expression cassettes of the invention can further comprise enhancer elements and/or tissue preferred elements in combination with the promoter.

Further, in some embodiments, it is advantageous for the expression cassette to comprise a selectable marker gene for the selection of transformed cells. Suitable selectable marker genes include without limitation genes encoding antibiotic resistance, such as those encoding neomycin phosphotransferase II (NEO) and hygromycin phosphotransferase (HPT), as well as genes conferring resistance to herbicidal compounds. Herbicide resistance genes generally code for a modified target protein insensitive to the herbicide or for an enzyme that degrades or detoxifies the herbicide in the plant before it can act. See, DeBlock et al., EMBO J. 6, 2513 (1987); DeBlock et al., Plant Physiol. 91, 691 (1989); Fromm et al., BioTechnology 8, 833 (1990); Gordon-Kamm et al., Plant Cell 2, 603 (1990). For example, resistance to glyphosphate or sulfonylurea herbicides has been obtained using genes coding for the mutant target enzymes, 5-enolpyruvylshikimate-3-phosphate synthase (EPSPS) and acetolactate synthase (ALS). Resistance to glufosinate ammonium, boromoxynil, and 2,4-dichlorophenoxyacetate (2,4-D) have been obtained by using bacterial genes encoding phosphinothricin acetyltransferase, a nitrilase, or a 2,4-dichlorophenoxyacetate monooxygenase, which detoxify the respective herbicides.

Selectable marker genes that can be used according to the present invention further include, but are not limited to, genes encoding: neomycin phosphotransferase II (Fraley et al., CRC Critical Reviews in Plant Science 4, 1 (1986)); cyanamide hydratase (Maier-Greiner et al., Proc. Natl. Acad. Sci. USA 88, 4250 (1991)); aspartate kinase; dihydrodipicolinate synthase (Perl et al., BioTechnology 11, 715 (1993)); the bar gene (Toki et al., Plant Physiol. 100, 1503 (1992); Meagher et al., Crop Sci. 36, 1367 (1996)); tryptophane decarboxylase (Goddijn et al., Plant Mol. Biol. 22, 907 (1993)); neomycin phosphotransferase (NEO; Southern et al., J. Mol. Appl. Gen. 1, 327 (1982)); hygromycin phosphotransferase (HPT or HYG; Shimizu et al., Mol. Cell. Biol. 6, 1074 (1986)); dihydrofolate reductase (DHFR; Kwok et al., Proc. Natl. Acad. Sci. USA 83, 4552 (1986)); phosphinothricin acetyltransferase (DeBlock et al., EMBO J. 6, 2513 (1987)); 2,2-dichloropropionic acid dehalogenase (Buchanan-Wollatron et al., J. Cell. Biochem. 13D, 330 (1989)); acetohydroxyacid synthase (U.S. Pat. No. 4,761,373 to Anderson et al.; Haughn et al., Mol. Gen. Genet. 221, 266 (1988)); 5-enolpyruvyl-shikimate-phosphate synthase (aroA; Comai et al., Nature 317, 741 (1985)); haloarylnitrilase (WO 87/04181 to Stalker et al.); acetyl-coenzyme A carboxylase (Parker et al., Plant Physiol. 92, 1220 (1990)); dihydropteroate synthase (sulI; Guerineau et al., Plant Mol. Biol. 15, 127 (1990)); and 32 kDa photosystem II polypeptide (psbA; Hirschberg et al., Science 222, 1346 (1983)).

Also included are genes encoding resistance to: chloramphenicol (Herrera-Estrella et al., EMBO J. 2, 987 (1983)); methotrexate (Herrera-Estrella et al., Nature 303, 209 (1983); Meijer et al., Plant Mol. Biol. 16, 807 (1991)); hygromycin (Waldron et al., Plant Mol. Biol. 5,103 (1985); Zhijian et al., Plant Science 108, 219 (1995); Meijer et al., Plant Mol. Bio. 16, 807 (1991)); streptomycin (Jones et al., Mol. Gen. Genet. 210, 86 (1987)); and spectinomycin (Bretagne-Sagnard et al., Transgenic Res. 5, 131 (1996)); bleomycin (Hille et al., Plant Mol. Biol. 7, 171 (1986)); sulfonamide (Guerineau et al., Plant Mol. Bio. 15, 127 (1990); bromoxynil (Stalker et al., Science 242, 419 (1988)); 2,4-D (Streber et al., Bio/Technology 7, 811 (1989)); phosphinothricin (DeBlock et al., EMBO J. 6, 2513 (1987)); spectinomycin (Bretagne-Sagnard and Chupeau, Transgenic Research 5, 131 (1996)).

Other selectable marker genes include the pat gene (for bialaphos and phosphinothricin resistance), the ALS gene for imidazolinone resistance, the HPH or HYG gene for hygromycin resistance, the Hm1 gene for resistance to the Hc-toxin, and other selective agents used routinely and known to one of ordinary skill in the art. See generally, Yarranton, Curr. Opin. Biotech, 3, 506 (1992); Chistopherson et al., Proc. Natl. Acad. Sci, USA 89, 6314 (1992); Yao et al., Cell 71, 63 (1992); Reznikoff, Mol. Microbiol. 6, 2419 (1992); BARKLEY ET AL., THE OPERON 177-220 (1980); Hu et al., Cell 48, 555 (1987); Brown et al., Cell 49, 603 (1987); Figge et al., Cell 52, 713 (1988); Deuschle et al., Proc. Natl. Acad. Sci. USA 86, 5400 (1989); Fuerst et al., Proc. Natl. Acad. Sci. USA 86, 2549 (1989); Deuschle et al., Science 248, 480 (1990); Labow et al., Mol. Cell. Biol. 10, 3343 (1990); Zambretti et al., Proc. Natl. Acad. Sci. USA 89, 3952 (1992); Baim et al., Proc. Natl. Acad. Sci. USA 88, 5072 (1991); Wyborski et al., Nuc. Acids Res. 19, 4647 (1991); Hillenand-Wissman, Topics in Mol. And Struc. Biol. 10, 143 (1989); Degenkolb et al., Antimicrob. Agents Chemother. 35, 1591 (1991); Kleinschnidt et al., Biochemistry 27, 1094 (1988); Gatz et al., Plant J. 2, 397 (1992); Gossen et al., Proc. Natl. Acad. Sci. USA 89, 5547 (1992); Oliva et al., Antimicrob. Agents Chemother. 36, 913 (1992); Hlavka et al., Handbook of Experimental Pharmacology 78 (1985); and Gill et al., Nature 334, 721 (1988).

A polynucleotide of interest can additionally be operably linked to a sequence that encodes a transit peptide that directs expression of an encoded polypeptide of interest to a particular cellular compartment. Transit peptides that target protein accumulation in higher plant cells to the chloroplast, mitochondrion, vacuole, nucleus, and the endoplasmic reticulum (for secretion outside of the cell) are known in the art. Transit peptides that target proteins to the endoplasmic reticulum are desirable for correct processing of secreted proteins. Targeting protein expression to the chloroplast (for example, using the transit peptide from the RubP carboxylase small subunit gene) has been shown to result in the accumulation of very high concentrations of recombinant protein in this organelle. The pea RubP carboxylase small subunit transit peptide sequence has been used to express and target mammalian genes in plants (U.S. Pat. Nos. 5,717,084 and 5,728,925 to Herrera-Estrella et al.). Alternatively, mammalian transit peptides can be used to target recombinant protein expression, for example, to the mitochondrion and endoplasmic reticulum. It has been demonstrated that plant cells recognize mammalian transit peptides that target endoplasmic reticulum (U.S. Pat. Nos. 5,202,422 and 5,639,947 to Hiatt et al.).

Further, the expression cassette can comprise a 5′ leader sequence that acts to enhance expression (transcription, post-transcriptional processing and/or translation) of an operably associated nucleotide sequence of interest. Leader sequences are known in the art and include sequences from: picornavirus leaders, e.g., EMCV leader (Encephalomyocarditis 5′ noncoding region; Elroy-Stein et al., Proc. Natl. Acad, Sci USA, 86, 6126 (1989)); potyvirus leaders, e.g., TEV leader (Tobacco Etch Virus; Allison et al., Virology, 154, 9 (1986)); human immunoglobulin heavy-chain binding protein (BiP; Macajak and Sarnow, Nature 353, 90 (1991)); untranslated leader from the coat protein mRNA of alfalfa mosaic virus (AMV RNA 4; Jobling and Gehrke, Nature 325, 622 (1987)); tobacco mosaic virus leader (TMV; Gallie, Molecular Biology of RNA, 237-56 (1989)); and maize chlorotic mottle virus leader (MCMV; Lommel et al., Virology 81, 382 (1991)). See also, Della-Cioppa et al., Plant Physiology 84, 965 (1987).

### II. Polynucleotides of Interest.

The polynucleotide(s) of interest in the expression cassette can be any polynucleotide(s) of interest and can be obtained from prokaryotes or eukaryotes (e.g., bacteria, fungi, yeast, viruses, plants, mammals) or the polynucleotide of interest can be synthesized in whole or in part. Further, the polynucleotide of interest can encode a polypeptide of interest or can be transcribed to produce a functional RNA. In particular embodiments, the functional RNA can be expressed to improve an agronomic trait in the plant (e.g., tolerance to drought, heat stress, high temperature, low pH, salt, or resistance to pollutants, heavy metals, herbicides, disease-causing organisms (e.g., fungi, bacteria, viruses), insects or other pests [e.g., a Bacillus thuringiensis endotoxin], and the like), to confer male sterility, to improve mineral/soil nutrient uptake (e.g. nitrogen, phosphate, other macro-nutrients and micro-nutrients) or to improve communication with beneficial microbes. A polypeptide of interest can be any polypeptide encoded by a polynucleotide sequence of interest. The polynucleotide sequence may further be used in the sense orientation to achieve suppression of endogenous plant genes, as is known by those skilled in the art (see, e.g., U.S. Pat. Nos. 5,283,184; 5,034,323).

The nucleotide sequence of interest can encode a polypeptide that imparts a desirable agronomic trait to the plant (as described above), confers male sterility, improves fertility and/or improves nutritional quality. Other suitable polypeptides include enzymes that can degrade organic pollutants or remove heavy metals. Such plants, and the enzymes that can be isolated therefrom, are useful in methods of environmental protection and remediation. Alternatively, the heterologous nucleotide sequence can encode a therapeutically or pharmaceutically useful polypeptide or an industrial polypeptide (e.g., an industrial enzyme) Therapeutic polypeptides include, but are not limited to antibodies and antibody fragments, cytokines, hormones, growth factors, receptors, enzymes and the like.

Additional non-limiting examples of polypeptides of interest that are suitable for use with this invention (e.g., to be expressed in response to exposure to nitrate, drought, and/or rehydration) include polypeptides associated with nutrient uptake including transport and assimilation of organic and inorganic nutrients. Thus, for example, polypeptides involved in nitrogen transport and assimilation, including but not limited to, nitrite transporter (NiTR1 gene), high affinity nitrate transporter, nitrate and chloride transporter, nitrate reductase (nr2), NADH-dependent nitrate reductase, oligopeptide and nitrate transporter, ammonium transporter (Osamt1.1; 1.3; 2.2; 3.1; 5.1), nitrate transporter (Atnt1 1), symbiotic ammonium transporter, ammonium transporter, NADH-dependent glutamate synthase, nitrate transporter, ammonium transporter (Osamt1.1; 5.2), high affinity nitrate transporter (nar2.1), gln4, g15, nitrate transporter (nrt1.1), amino acid transport protein, NADH-dependent nitrate reductase (nr1), nitrate transporter (nrt1-5), ammonium transporter (Osamt2.1; 2.3; 3.3), high affinity nitrate transporter (nar2.1; nar2.2), nitrate transporter (Glycine max nrt1.2), ferredoxin-dependent glutamate synthase, high affinity nitrate transporter (nrt2.1)

Other non-limiting examples of polypeptides of interest include those involved in resistance to insects, nematodes and pathogenic diseases. Such polypeptides can include but are not limited to glucosinolates (defense against herbivores), chitinases or glucanases and other enzymes which destroy the cell wall of parasites, ribosome-inactivating proteins (RIPs) and other proteins of the plant resistance and stress reaction as are induced when plants are wounded or attacked by microbes, or chemically, by, for example, salicylic acid, jasmonic acid or ethylene, or lysozymes from nonplant sources such as, for example, T4-lysozyme or lysozyme from a variety of mammals, insecticidal proteins such as Bacillus thuringiensis endotoxin, a-amylase inhibitor or protease inhibitors (cowpea trypsin inhibitor), lectins such as wheatgerm agglutinin, RNAses or ribozymes. Further non-limiting examples include nucleic acids which encode the Trichoderma harzianum chit42 endochitinase (GenBank Ace. No.: 578423) or the N-hydroxylating, multi-functional cytochrome P-450 (CYP79) protein from Sorghum bicolor (GenBank Ace. No.: U32624), or functional equivalents of these, chitinases, for example from beans (Brogue et al. (1991) Science 254:1194-1197), “polygalacturonase-inhibiting protein” (PGIP), thaumatine, invertase and antimicrobial peptides such as lactoferrin (Lee T J et al. (2002) J Amer Soc Horticult Sci 127(2):158-164) (See, e.g., U.S. Pat. No. 8,071,749) as well as the plant defense genes, including but not limited to, PR1, BG2, PR5, and NPR1 (or NIM1).

Also useful with the present invention are nucleotide sequences encoding polypeptides involved in plant hormone production or signaling including, but not limited to, auxins, cytokinins, gibberellins, strigolactones, ethylene, jasmonic acid, and brassinosteroids, as well as other nucleotide and polypeptide sequences that regulate or effect root and leaf growth and development. Non-limiting examples of such nucleotide and/or polypeptide sequences include GA-Deficient-1 (GA1; CPS), Gibberellin 20-Oxidase (GA20ox, GA5 (in At)), Gibberellin 2-beta-dioxygenase (GA2ox), Gibberellin 3-Oxidase (GA3ox), GA-Insensitive (GAI),GA Regulated MYB(GAMYB), GCA2 Growth Controlled By ABA 2 (GCA2), G-Protein Coupled Receptor (GCR1), Glycosyl Hydrolase Family-45 (GH45), tryptophan synthase alpha chain (e.g.,GRMZM2G046163, GRMZM2G015892), Auxin Binding Protein 1 (ABP1), IAA-amino acid hydrolase ILR1 (e.g., GRMZM2G091540), phosphoribosylanthranilate transferase, Indole Acetic Acid 17/Auxin Resistant 3(IAA17, AXR3), Indole Acetic Acid 3/Short Hypocotyl (IAA3, SHY2), IAA-lysine synthetase (iaaL), tryptophan monooxygenase (iaaM), IAA-Aspartic Acid Hydrolase (IaaspH), IAA-Glucose Synthase (IAGLU),IndoleAcetamide Hydrolase (IAH), Indole-3-Acetaldehyde Oxidase (IAO),IAA-ModifiedProtein (IAP1), Auxin Response factors (ARFs), small auxin up RNA (SAUR), Induced By Cytokinin 6 (Same as ARR5)(IBC6), Induced By Cytokinin 7 (Same as ARR4) IBC7, Viviparous-14 (Vp14), PLA₂ (Zhu J-K. Annual Review of Plant Biology 2002, 53(1):247-273), ATPLC2 (Benschop et al. Plant Physiology 2007, 143(2):1013-1023), inositol polyphosphate 5-phosphatase (At5PTaseI), calcium-dependent protein kinases (CDPKs), calcineurin B-like (CBL) calcium sensor protein CBL4/SOS3, CIPK-like protein 1, ACC (1-aminocyclopropane-1-carboxylate) synthase, ACC oxidase, phosphatase 2C ABI1, TINY, maize lipoxygenase 7 (GRMZM2G070092), allene oxide synthase (AOS) (e.g., GRMZM2G033098 and GRMZM2G376661), short chain alcohol dehydrogenases (ADH), Tasselseed2 (Ts2), Tasselseed1 (Ts1), Supercentipede1 (Scn1/GDI1,e.g., AT2G44100), RDH2 (Carol et al. Nature 2005, 438(7070):1013-1016.), G-signaling proteins, Morphogenesis of Root Hair (MRH), AtAGC2-1 (e.g., At3g25250), Cellulose Synthase-Like D3 (CSLD3), xylosyltransferase 2 (e.g., At4g02500, AtXX2), xyloglucan endotransglucosylase/hydrolase 26 (e.g., AtXTH26, At4g28850), xyloglucan endotransglycosylase, xyloglucan galact-osyltransferase (MUR3 (e.g.,AT2G20370), ARP2/3 (WURM/DISTORTED 1) complex, and germin-like protein (e.g., AT5G39110).

Other nucleotide sequences and polypeptides that are suitable for use with the present invention include those that confer the “stay-green” phenotype (See, Hortensteiner, S. Trends in Plant Science 14: 155-162 (2009)). Non-limiting examples of such nucleotide sequences include MtSGR, MsSGR (Zhou et al. Plant Physiol. 157: 1483-1496 (2011)), STAY-GREEN (SGR or SGN) (Jiang et al., Plant J 52: 197-209 (2007)), Park et al., Plant Cell 19: 1649-1664 (2007)), NONYELLOWING (NYE1) (Ren et al., Plant Physiol 144: 1429-1441 (2007)), and/or GREEN-FLESH (GF) or CHLOROPHYLL RETAINER (CL) (Barry et al., Plant Physiol 147: 179-187 (2008)).

Polynucleotides involved in grain filling are also useful with the present invention and include, but are not limited to GIF1 (GRAIN INCOMPLETE FILLING 1) from rice.

Other non-limiting examples of polypeptides of interest that are suitable for production in plants include those resulting in agronomically important traits such as herbicide resistance (also sometimes referred to as “herbicide tolerance”), virus resistance, bacterial pathogen resistance, insect resistance, nematode resistance, and/or fungal resistance. See, e.g., U.S. Pat. Nos. 5,569,823; 5,304,730; 5,495,071; 6,329,504; and 6,337,431. The polypeptide also can be one that increases plant vigor or yield (including traits that allow a plant to grow at different temperatures, soil conditions and levels of sunlight and precipitation), or one that allows identification of a plant exhibiting a trait of interest (e.g., a selectable marker, seed coat color, etc.). Various polypeptides of interest, as well as methods for introducing these polypeptides into a plant, are described, for example, in U.S. Pat. Nos. 4,761,373; 4,769,061; 4,810,648; 4,940,835; 4,975,374; 5,013,659; 5,162,602; 5,276,268; 5,304,730; 5,495,071; 5,554,798; 5,561,236; 5,569,823; 5,767,366; 5,879,903, 5,928,937; 6,084,155; 6,329,504 and 6,337,431; as well as US Patent Publication No. 2001/0016956. See also, on the World Wide Web at lifesci.sussex.ac.uk/home/Neil_Crickmore/Bt/.

Nucleotide sequences conferring resistance/tolerance to an herbicide that inhibits the growing point or meristem, such as an imidazalinone or a sulfonylurea can also be suitable in some embodiments of the invention. Exemplary nucleotide sequences in this category code for mutant ALS and AHAS enzymes as described, e.g., in U.S. Pat. Nos. 5,767,366 and 5,928,937. U.S. Pat. Nos. 4,761,373 and 5,013,659 are directed to plants resistant to various imidazalinone or sulfonamide herbicides. U.S. Pat. No. 4,975,374 relates to plant cells and plants containing a nucleic acid encoding a mutant glutamine synthetase (GS) resistant to inhibition by herbicides that are known to inhibit GS, e.g., phosphinothricin and methionine sulfoximine. U.S. Pat. No. 5,162,602 discloses plants resistant to inhibition by cyclohexanedione and aryloxyphenoxypropanoic acid herbicides. The resistance is conferred by an altered acetyl coenzyme A carboxylase (ACCase).

In embodiments of the invention, the nucleotide sequence increases tolerance of a plant, plant part and/or plant cell to heat stress and/or high temperature. The nucleotide sequence can encode a polypeptide or inhibitory polynucleotide (e.g., functional RNA) that results in increased tolerance to heat stress and/or high temperature. Suitable polypeptide include without limitation water stress polypeptides, ABA receptors, and dehydration proteins (e.g., dehydrins (ERDs)).

In representative embodiments, nucleotide sequences that encode polypeptides that provide tolerance to water stress (e.g., drought) are used. Non-limiting examples of polypeptides that provide tolerance to water stress include: water channel proteins involved in the movement of water through membranes; enzymes required for the biosynthesis of various osmoprotectants (e.g., sugars, proline, and Glycine-betaine); proteins that protect macromolecules and membranes (e.g., LEA protein, osmotin, antifreeze protein, chaperone and mRNA binding proteins); proteases for protein turnover (thiol proteases, Clp protease and ubiquitin); and detoxification enzymes (e.g., glutathione S-transferase, soluble epoxide hydrolase, catalase, superoxide dismutase and ascorbate peroxidase). Non-limiting examples of proteins involved in the regulation of signal transduction and gene expression in response to water stress include protein kinases (MAPK, MAPKKK, S6K, CDPK, two-component His kinase, Bacterial-type sensory kinase and SNF1); transcription factors (e.g., MYC and bZIP); phosopholipase C; and 14-3-3 proteins.

Nucleotide sequences that encode receptors/binding proteins for abscisic acid (ABA) are also useful in the practice of the present invention. Non-limiting examples of ABA binding proteins/receptors include: the Mg-chelatase H subunit; RNA-binding protein FCA; G-protein coupled receptor GCR2; PYR1; PYL5; protein phosphatases 2C ABI1 and ABI2; and proteins of the RCAR (Regulatory Component of the ABA Receptor) family.

In embodiments of the invention, the nucleotide sequence encodes a dehydration protein, also known as a dehydrin (e.g., an ERD). Dehyration proteins are a group of proteins known to accumulate in plants in response to dehydration. Examples include WCOR410 from wheat; PCA60 from peach; DHN3 from sessile oak, COR47 from Arabidopsis thaliana; Hsp90, BN59, BN115 and Bnerd10 from Brassica napes; COR39 and WCS19 from Triticum aestivum (bread wheat); and COR25 from Brassica rapa subsp. Pekinensis. Other examples of dehydration proteins are ERD proteins, which include without limitation, ERD1, ERD2, ERD4, ERD5, ERD6, ERD8, ERD10, ERD11, ERD13, ERD15 and ERD16.

Polypeptides encoded by nucleotide sequences conferring resistance to glyphosate are also suitable for use with the present invention. See, e.g., U.S. Pat. No. 4,940,835 and U.S. Pat. No. 4,769,061. U.S. Pat. No. 5,554,798 discloses transgenic glyphosate resistant maize plants, which resistance is conferred by an altered 5-enolpyruvyl-3-phosphoshikimate (EPSP) synthase gene. Heterologous nucleotide sequences suitable to confer tolerance to the herbicide glyphosate also include, but are not limited to the Agrobacterium strain CP4 glyphosate resistant EPSPS gene (aroA:CP4) as described in U.S. Pat. No. 5,633,435 or the glyphosate oxidoreductase gene (GOX) as described in U.S. Pat. No. 5,463,175. Other heterologous nucleotide sequences include genes conferring resistance to herbicides that act to inhibit the action of acetolactate synthase (ALS), in particular the sulfonylurea-type herbicides (e.g., mutant forms of the acetolactate synthase (ALS) gene that lead to such resistance, in particular the S4 and/or Hra mutations), genes coding for resistance to herbicides that act to inhibit the action of glutamine synthase, such as phosphinothricin or basta (e.g., the bar gene). The bar gene encodes resistance to the herbicide basta, the nptII gene encodes resistance to the antibiotics kanamycin and geneticin, and the ALS gene encodes resistance to the herbicide chlorsulfuron.

Nucleotide sequences coding for resistance to phosphono compounds such as glufosinate ammonium or phosphinothricin, and pyridinoxy or phenoxy propionic acids and cyclohexones are also suitable. See, European Patent Application No. 0 242 246. See also, U.S. Pat. Nos. 5,879,903, 5,276,268 and 5,561,236.

Other suitable nucleotide sequences of interest include those coding for resistance to herbicides that inhibit photosynthesis, such as a triazine and a benzonitrile (nitrilase). See, U.S. Pat. No. 4,810,648. Additional suitable nucleotide sequences coding for herbicide resistance include those coding for resistance to 2,2-dichloropropionic acid, sethoxydim, haloxyfop, imidazolinone herbicides, sulfonylurea herbicides, triazolopyrimidine herbicides, s-triazine herbicides and bromoxynil. Also suitable are nucleotide sequences conferring resistance to a protox enzyme, or that provide enhanced resistance to plant diseases; enhanced tolerance of adverse environmental conditions (abiotic stresses) including but not limited to drought, heat stress, high temperature, cold, excessive soil salinity or extreme acidity or alkalinity; and alterations in plant architecture or development, including changes in developmental timing. See, e.g., U.S. Patent Publication No. 2001/0016956 and U.S. Pat. No. 6,084,155.

Insecticidal proteins useful in the invention may be produced in an amount sufficient to control insect pests, i.e., insect controlling amounts. It is recognized that the amount of production of insecticidal protein in a plant useful to control insects may vary depending upon the cultivar, type of insect, environmental factors and the like. Suitable heterologous nucleotide sequences that confer insect tolerance include those which provide resistance to pests such as rootworm, cutworm, European Corn Borer, and the like. Exemplary nucleotide sequences include, but are not limited to, those that encode toxins identified in Bacillus organisms (see, e.g., WO 99/31248; U.S. Pat. Nos. 5,689,052; 5,500,365; 5,880,275); Bacillus thuringiensis toxic protein genes (see, e.g., U.S. Pat. Nos. 5,366,892; 5,747,450; 5,737,514; 5,723,756; 5,593,881; 6,555,655; 6,541,448; 6,538,109; Geiser, et al. (1986) Gene 48:109); and lectins (Van Damme et al. (1994) Plant Mol. Biol, 24:825). Nucleotide sequences encoding Bacillus thuringiensis (Bt) toxins from several subspecies have been cloned and recombinant clones have been found to be toxic to lepidopteran, dipteran and coleopteran insect larvae (for example, various delta-endotoxin genes such as Cry1Aa, Cry1Ab, Cry1Ac, Cry1B, Cry1C, Cry1D, Cry1Ea, Cry1Fa, Cry3A, Cry9A, Cry9C and Cry9B; as well as genes encoding vegetative insecticidal proteins such as Vip1, Vip2 and Vip3). A full list of Bt toxins can be found on the worldwide web at Bacillus thuringiensis Toxin Nomenclature Database maintained by the University of Sussex (see also, Crickmore et al. (1998) Microbiol. Mol. Biol. Rev. 62:807-813).

Polypeptides that are suitable for production in plants further include those that improve or otherwise facilitate the conversion of harvested plants and/or plant parts into a commercially useful product, including, for example, increased or altered carbohydrate content and/or distribution, improved fermentation properties, increased oil content, increased protein content, improved digestibility, and increased nutraceutical content, e.g., increased phytosterol content, increased tocopherol content, increased stanol content and/or increased vitamin content. Polypeptides of interest also include, for example, those resulting in, or contributing to, a reduced content of an unwanted component in a harvested crop; e.g., phytic acid, or sugar degrading enzymes. By “resulting in” or “contributing to” is intended that the polypeptide of interest can directly or indirectly contribute to the existence of a trait of interest (e.g., increasing cellulose degradation by the use of a heterologous cellulase enzyme).

In one embodiment, the polypeptide of interest contributes to improved digestibility for food or feed. Xylanases are hemicellulolytic enzymes that improve the breakdown of plant cell walls, which leads to better utilization of the plant nutrients by an animal. This leads to improved growth rate and feed conversion. Also, the viscosity of the feeds containing xylan can be reduced by xylanases. Heterologous production of xylanases in plant cells also can facilitate lignocellulosic conversion to fermentable sugars in industrial processing.

Numerous xylanases from fungal and bacterial microorganisms have been identified and characterized (see, e.g., U.S. Pat. No. 5,437,992; Coughlin et al. (1993) “Proceedings of the Second TRICEL Symposium on Trichoderma reesei Cellulases and Other Hydrolases” Espoo; Souminen and Reinikainen, eds. (1993) Foundation for Biotechnical and Industrial Fermentation Research 8:125-135; U.S. Patent Publication No. 2005/0208178; and PCT Publication No. WO 03/16654). In particular, three specific xylanases (XYL-I, XYL-II, and XYL-III) have been identified in T. reesei (Tenkanen et al. (1992) Enzyme Microb. Technol. 14:566; Torronen et al. (1992) Bio/Technology 10:1461; and Xu et al. (1998) Appl. Microbiol. Biotechnol. 49:718).

In another embodiment, a polypeptide useful for the present invention can be a polysaccharide degrading enzyme. Plants producing such an enzyme may be useful for generating, for example, fermentation feedstocks for bioprocessing. In some embodiments, enzymes useful for a fermentation process include alpha amylases, proteases, pullulanases, isoamylases, cellulases, hemicellulases, xylanases, cyclodextrin glycotransferases, lipases, phytases, laccases, oxidases, esterases, cutinases, granular starch hydrolyzing enzyme or other glucoamylases.

Polysaccharide-degrading enzymes include: starch degrading enzymes such as alpha-amylases (EC 3.2.1.1), glucuronidases (E.C. 121131), exo-1,4-alpha-D glucanases such as amyloglucosidases and glucoamylase (EC 3.2.1.3), beta-amylases (EC 3.2.1.2), alpha-glucosidases (EC 3.2.1.20), and other exo-amylases, starch debranching enzymes, such as a) isoamylase (EC 3.2.1.68), pullulanase (EC 3.2.1.41), and the like; b) cellulases such as exo-1,4-3-cellobiohydrolase (EC 12191), exo-1,3-beta-D-glucanase (EC 3.2.1.39), beta-glucosidase (EC 3.2.1.21); c) L-arabinases, such as endo-1,5-alpha-L-arabinase (EC 3.2.1.99), alpha-arabinosidases (EC 3.2.1.55) and the like; d) galactanases such as endo-1,4-beta-D-galactanase (EC 3.2.1.89), endo-1,3-beta-D-galactanase (EC 3.2.1.90), alpha-galactosidase (EC 3.2.1.22), beta-galactosidase (EC 3.2.1.23) and the like; e) mannanases, such as endo-1,4-beta-D-mannanase (EC 3.2.1.78), beta-mannosidase (EC 3.2.1.25), alpha-mannosidase (EC 3.2.1.24) and the like; f) xylanases, such as endo-1,4-beta-xylanase (EC 3.2.1.8), beta-D-xylosidase (EC 3.2.1.37), 1,3-beta-D-xylanase, and the like; and g) other enzymes such as alpha-L-fucosidase (EC 12151), alpha-L-rhamnosidase (EC 3.2.1.40), levanase (EC 3.2.1.65), inulanase (EC 3.2.1.7), and the like.

Further enzymes which may be used with the present invention include proteases, such as fungal and bacterial proteases. Fungal proteases include, but are not limited to, those obtained from Aspergillus, Trichoderma, Mucor and Rhizopus, such as A. niger, A. awamori, A. oryzae and M miehei.

Other useful enzymes include, but are not limited to, hemicellulases, such as mannases and arabinofuranosidases (EC 3.2.1.55); ligninases; lipases (e.g., E.C. 3.1.1.3), glucose oxidases, pectinases, xylanases, transglucosidases, alpha 1,6 glucosidases (e.g., E.C. 3.2.1.20); cellobiohydrolases; esterases such as ferulic acid esterase (EC 3.1.1.73) and acetyl xylan esterases (EC 3.1.1.72); and cutinases (e.g. E.C. 3.1.1.74).

The nucleotide sequence can encode a reporter polypeptide (e.g., an enzyme), including but not limited to Green Fluorescent Protein, β-galactosidase, luciferase, alkaline phosphatase, the GUS gene encoding β-glucuronidase, and chloramphenicol acetyltransferase.

Where appropriate, the nucleotide sequence of interest may be optimized for increased expression in a transformed plant, e.g., by using plant preferred codons. Methods for synthetic optimization of nucleic acid sequences are available in the art. The nucleotide sequence of interest can be optimized for expression in a particular host plant or alternatively can be modified for optimal expression in monocots. See, e.g., EP 0 359 472, EP 0 385 962, WO 91/16432; Perlak et al., Proc. Natl. Acad. Sci. USA 88, 3324 (1991), and Murray et al., Nuc. Acids Res. 17, 477 (1989), and the like. Plant preferred codons can be determined from the codons of highest frequency in the proteins expressed in that plant.

Additional sequence modifications are known to enhance gene expression in a cellular host. These include elimination of sequences encoding spurious polyadenylation signals, exon-intron splice site signals, transposon-like repeats, and other such well-characterized sequences which may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

### III. Transgenic Plants, Plant Parts and Plant Cells.

The invention also provides transgenic plants, plant parts and plant cells comprising the nucleic acids, expression cassettes and vectors of the invention.

Accordingly, one aspect the invention provides a cell comprising a nucleic acid, expression cassette, or vector of the invention. The cell can be transiently or stably transformed with the nucleic acid, expression cassette and/or vector. Further, the cell can be a cultured cell, a cell obtained from a plant, plant part, or plant tissue, or a cell in situ in a plant, plant part or plant tissue. Cells can be from any suitable species, including plant (e.g., corn), bacterial, yeast, insect and/or mammalian cells. In representative embodiments, the cell is a plant cell or bacterial cell.

The invention also provides a plant part (including a plant tissue culture) comprising a nucleic acid, expression cassette, or vector of the invention. The plant part can be transiently or stably transformed with the nucleic acid, expression cassette or vector. Further, the plant part can be in culture, can be a plant part obtained from a plant, or a plant part in situ. In representative embodiments, the plant part comprises a cell of the invention.

Seed comprising the nucleic acid, expression cassette, or vector of the invention are also provided. In some embodiments of the present invention, the nucleic acid, expression cassette or vector is stably incorporated into the genome of the seed.

The invention also contemplates a transgenic plant comprising a nucleic acid, expression cassette, and/or vector of the invention. The plant can be transiently or stably transformed with a nucleic acid, expression cassette or vector comprising a recombinant promoter sequence of the invention. In representative embodiments, the plant comprises a cell or plant part of the invention (as described above). In representative embodiments, a promoter comprising, consisting essentially of, or consisting of a nucleotide sequence of the invention (e.g., SEQ ID NO:1, SEQ ID NO:2, SEQ ID NO:3, SEQ ID NO:9, SEQ ID NO:10, SEQ ID SEQ ID NO:12, SEQ ID NO:13, SEQ ID NO:14, SEQ ID NO:15, SEQ ID NO:16, SEQ ID NO:17, SEQ ID NO:18, SEQ ID NO:19, and/or SEQ ID NO:20) is inducible (e.g., has increased activity) in response to nitrate, drought, and/or rehydration. In other embodiments, a promoter comprising, consisting essentially of, or consisting of a nucleotide sequence of the invention (e.g., SEQ ID SEQ ID NO:2, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, and/or SEQ ID NO:20) is repressible (e.g., has decreased activity) in response to nitrate, drought, and/or rehydration.

Still further, the invention encompasses a crop comprising a plurality of the transgenic plants of the invention, as described herein. Nonlimiting examples of the types of crops comprising a plurality of transgenic plants of the invention include an agricultural field, a golf course, a residential lawn or garden, a public lawn or garden, a road side planting, an orchard, and/or a recreational field (e.g., a cultivated area comprising a plurality of the transgenic plants of the invention).

Products harvested from the plants of the invention are also provided. Nonlimiting examples of a harvested product include a seed, a leaf, a stem, a shoot, a fruit, flower, root, biomass (e.g., for biofuel production) and/or extract.

In some embodiments, a processed product produced from the harvested product is provided. Nonlimiting examples of a processed product include a polypeptide (e.g., a recombinant polypeptide), an extract, a medicinal product (e.g., artemicin as an antimalarial agent), a fiber or woven textile, a fragrance, dried fruit, a biofuel (e.g., ethanol), a tobacco product (e.g., cured tobacco, cigarettes, chewing tobacco, cigars, and the like), an oil (e.g., sunflower oil, corn oil, canola oil, and the like), a nut or seed butter, a flour or meal (e.g., wheat or rice flour, corn meal) and/or any other animal feed (e.g., soy, maize, barley, rice, alfalfa) and/or human food product (e.g., a processed wheat, maize, rice or soy food product).

### IV. Methods of Introducing Nucleic Acids.

The invention also provides methods of introducing a nucleic acid, expression cassette and/or vector as described herein into a target plant, plant part or plant cell (including callus cells or protoplasts), seed, plant tissue (including callus), and the like. In exemplary embodiments, the method is practiced to express a nucleotide sequence of interest that is operably associated with a promoter comprising, consisting essentially of, or consisting of a nucleotide sequence of the present invention (e.g., SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, and/or SEQ ID NO:20, in any combination) as described herein. As described herein, the nucleotide sequences of the invention can be used in any combination. Thus, in some embodiments, a recombinant promoter can comprise, consist essentially of, or consist of one or more different nucleotide sequences of the invention, one or more of the same nucleotide sequence of the invention, or any combination thereof of the same or different nucleotide sequences of the invention. The invention further comprises plants (and progeny thereof), plant parts, seed, tissue culture (including callus) or cells, transiently or stably transformed with the nucleic acids, expression cassettes and/or vectors as described herein.

In representative embodiments, the invention provides a method of producing a plant comprising a nucleic acid of this invention, an expression cassette of this invention and/or a vector of this invention, the method comprising: introducing into a plant cell a nucleic acid of this invention, an expression cassette of this invention and/or a vector of this invention to produce a stably transformed plant cell; and regenerating a stably transformed plant from the plant cell.

In additional embodiments, the method comprises a method of expressing a polynucleotide of interest in a plant, the method comprising transforming a plant cell with an expression cassette or vector comprising a nucleic acid as described herein operably associated with a polynucleotide of interest to produce a transformed plant cell, regenerating a stably transformed transgenic plant from the transformed plant cell, and expressing the polynucleotide of interest in the plant.

Accordingly, in representative embodiments, a method of modulating the expression a polynucleotide of interest in a plant in response to nitrate (NO₃) is provided, the method comprising introducing into a plant cell a nucleic acid, expression cassette, and/or vector, wherein said nucleic acid, expression cassette, and/or vector comprises a recombinant promoter that comprises, consists essentially of, or consists of a nucleotide sequence of the invention (e.g., SEQ ID NO:1, SEQ ID NO:2, SEQ ID NO:3, SEQ ID NO:4, SEQ ID NO:5, SEQ ID NO:6, SEQ ID NO:7, and/or SEQ ID NO:8, in any combination) to produce a transformed plant cell; regenerating a transformed plant from the transformed plant cell; and exposing the transformed plant, or a plant part or plant cell therefrom, to NO₃, thereby modulating (i.e., increasing or decreasing) the expression a polynucleotide of interest in a plant in response to NO₃. In particular aspects of the invention, the nucleic acid, the expression cassette, and/or the vector comprise a recombinant promoter, wherein the recombinant promoter comprises, consists essentially of, or consists of one or more nucleotide sequences of SEQ ID NO:1, SEQ ID NO:2, SEQ ID NO:3, or any combination thereof, and the expression of the polynucleotide of interest is increased in response to nitrate as compared to the expression of the polynucleotide of interest when operably associated with the promoter that does not comprise a nucleotide sequence of SEQ ID No:1, SEQ ID NO:2, and/or SEQ ID NO:3. In other aspects of the invention, the nucleic acid, the expression cassette, and/or the vector comprise a promoter, wherein the promoter comprises, consists essentially of, or consists of one or more nucleotide sequences of SEQ ID NO:4, SEQ ID NO:5, SEQ ID NO:6, SEQ ID NO:7, SEQ ID NO:8, or any combination thereof, and the expression of the polynucleotide of interest is decreased in response to nitrate as compared to the expression of the polynucleotide of interest when operably associated with the promoter that does not comprise a nucleotide sequence of SEQ ID NO:4, SEQ ID NO:5, SEQ ID NO:6, SEQ ID NO:7, and/or SEQ ID NO:8.

In some embodiments, the plant is exposed to zero nitrate to about 30 mM nitrate. Thus, in further embodiments, the plant is exposed to about 0.001 mM, about 0.01 mM, about 0.1 mM, about 0.5 mM, about 1 mM, about 1.5 mM, about 2 mM, about 2.5 mM, about 3 mM, about 3.5 mM, about 4 mM, about 4.5 mM, about 6 mM, about 6.5 mM, 7 m M, about 7.5 mM, about 8 mM, about 9.5 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM nitrate, and the like, or any range therein. In some particular embodiments, the plant is exposed to about 0.3 mM nitrate to about 20 mM nitrate, about 0.3 mM nitrate to about 10 mM nitrate, about 0.3 mM nitrate to about 3 mM nitrate, about 1 mM nitrate to about 10 mM nitrate, and/or about 1 mM nitrate to about 5 mM nitrate.

In other embodiments, the promoter comprising, consisting of, or consisting essentially of a nucleotide sequence of the invention (e.g., SEQ ID NOs:1-20) is responsive to other nitrogen sources including but not limited to inorganic nitrogen, including but not limited to ammonium and nitrite, and organic nitrogen, including but not limited to, amino acids and peptides.

In another aspect of the invention, a method of modulating the expression of a polynucleotide of interest in a plant in response to drought is provided, the method comprising: introducing into a plant cell a nucleic acid of the invention, an expression cassette of the invention and/or a vector of the invention to produce a transformed plant cell; regenerating a transformed plant from the transformed plant cell; and exposing the transformed plant, or a plant part or plant cell therefrom, to drought, thereby modulating (i.e., increasing or decreasing) the expression a polynucleotide of interest in a plant in response to drought. In some aspects of the invention, the nucleic acid, the expression cassette, and/or the vector comprise a promoter, wherein the promoter comprises, consists essentially of, or consists of one or more nucleotide sequences of SEQ ID NO:9, SEQ ID NO:10, SEQ ID NO:19, SEQ ID NO:20, and any combination thereof, and the expression of the polynucleotide of interest is increased in response to drought as compared to the expression of the polynucleotide of interest when operably associated with the promoter that does not comprise a nucleotide sequence of SEQ ID NO:9, SEQ ID NO:10, SEQ ID NO:19, SEQ ID NO:20. In other aspects of the invention, the nucleic acid, the expression cassette, and/or the vector comprise a promoter, wherein the promoter comprises, consists essentially of, or consists of one or more nucleotide sequences of SEQ ID NO:11, SEQ ID NO:12, SEQ ID NO:13, SEQ ID NO:14, SEQ ID NO:15, SEQ ID NO:16, SEQ ID NO:17, SEQ ID NO:18, and any combination thereof, and the expression of the polynucleotide of interest is decreased in response to drought as compared to the expression of the polynucleotide of interest when operably associated with the promoter that does not comprise a nucleotide sequence of SEQ ID NO:11, SEQ ID NO:12, SEQ ID NO:13, SEQ ID NO:14, SEQ ID NO:15, SEQ ID NO:16, SEQ ID NO:17, SEQ ID NO:18.

A further aspect of the invention provides a method of modulating the expression of a polynucleotide of interest in a plant in response to rehydration, the method comprising:

introducing into a plant cell a nucleic acid of the invention, an expression cassette of the invention and/or a vector of the invention to produce a transformed plant cell; regenerating a transformed plant from the transformed plant cell; and rehydrating the transformed plant, or a plant part or plant cell therefrom, thereby modulating (i.e., increasing or decreasing) the expression a polynucleotide of interest in a plant in response to rehydration. In some aspects of the invention, the nucleic acid, the expression cassette, and/or the vector comprise a promoter, wherein the promoter comprises, consists essentially of, or consists of one or more nucleotide sequences of SEQ ID NO:9, SEQ ID NO:10, SEQ ID NO:19, SEQ ID NO:20, and any combination thereof, and the expression of the polynucleotide of interest is decreased in response to rehydration as compared to the expression of the polynucleotide of interest when operably associated with the promoter that does not comprise a nucleotide sequence of SEQ ID NO:9, SEQ ID NO:10, SEQ ID NO:19, SEQ ID NO:20. In other aspects of the invention, the nucleic acid, the expression cassette, and/or the vector comprise a promoter, wherein the promoter comprises, consists essentially of, or consists of one or more nucleotide sequences of SEQ ID NO:11, SEQ ID NO:12, SEQ ID NO:13, SEQ ID NO:14, SEQ ID NO:15, SEQ ID NO:16, SEQ ID NO:17, SEQ ID NO:18, and any combination thereof, and the expression of the polynucleotide of interest is increased in response to rehydration as compared to the expression of the polynucleotide of interest when operably associated with the promoter that does not comprise a nucleotide sequence of SEQ ID NO:11, SEQ ID NO:12, SEQ ID NO:13, SEQ ID NO:14, SEQ ID NO:15, SEQ ID NO:16, SEQ ID NO:17, SEQ ID NO:18.

Optionally, the methods of the invention can further comprise exposing the plant, plant part or plant cell to drought, rehydration and/or nitrate.

Thus, the present invention can be advantageously practiced to effect the expression of a polynucleotide of interest operably associated with a recombinant promoter as described herein, such that the polynucleotide sequence is expressed in response to one or more abiotic stimuli such as nitrate, drought, and/or rehydration. As described herein, the nucleotide sequences of the invention can be used in any combination. Further, the nucleotide sequences of the present invention can be used in combination with other promoter elements including but not limited to promoter motifs that confer developmental stage specific gene transcription. Thus, in some representative embodiments, in combination with promoter motifs that confer developmental stage specific gene transcription, the nucleotide sequences of the present invention can be used to provide plants that are, for example, drought tolerant at specific stages of development (e.g., juvenile, adult, reproductive, and the like).

The present invention further provides transgenic plants, plants parts including seed and progeny plants comprising the nucleic acids of this invention, crops comprising said plants, and harvested and processed products produced from the transgenic plants and plant parts of this invention.

Methods of introducing nucleic acids, transiently or stably, into plants, plant tissues, cells, protoplasts, seed, callus and the like are known in the art. Stably transformed nucleic acids can be incorporated into the genome. Exemplary transformation methods include biological methods using viruses and bacteria (e.g., Agrobacterium), physicochemical methods such as electroporation, floral dip methods, ballistic bombardment, microinjection, and the like. Other transformation technology includes the whiskers technology that is based on mineral fibers (see e.g., U.S. Pat. Nos. 5,302,523 and 5,464,765) and pollen tube transformation.

Other exemplary transformation methods include, without limitation, calcium-phosphate-mediated transformation, cyclodextrin-mediated transformation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

Thus, in some particular embodiments, the method of introducing into a plant, plant part, plant tissue, plant cell, protoplast, seed, callus and the like comprises bacterial-mediated transformation, particle bombardment transformation, calcium-phosphate-mediated transformation, cyclodextrin-mediated transformation, electroporation, liposome-mediated transformation, nanoparticle-mediated transformation, polymer-mediated transformation, virus-mediated nucleic acid delivery, whisker-mediated nucleic acid delivery, microinjection, sonication, infiltration, polyethyleneglycol-mediated transformation, any other electrical, chemical, physical and/or biological mechanism that results in the introduction of nucleic acid into the plant, plant part and/or cell thereof, or a combination thereof.

In one form of direct transformation, the vector is microinjected directly into plant cells by use of micropipettes to mechanically transfer the recombinant DNA (Crossway, Mol. Gen. Genetics 202: 179 (1985)).

In another protocol, the genetic material is transferred into the plant cell using polyethylene glycol (Krens, et al. Nature 296, 72 (1982)).

In still another method, protoplasts are fused with minicells, cells, lysosomes, or other fusible lipid-surfaced bodies that contain the nucleotide sequence to be transferred to the plant (Fraley, et al., Proc. Natl. Acad. Sci. USA 79, 1859 (1982)).

Nucleic acids may also be introduced into the plant cells by electroporation (Fromm et al., Proc. Natl. Acad. Sci. USA 82, 5824 (1985)). In this technique, plant protoplasts are electroporated in the presence of nucleic acids comprising the expression cassette. Electrical impulses of high field strength reversibly permeabilize biomembranes allowing the introduction of the nucleic acid. Electroporated plant protoplasts reform the cell wall, divide and regenerate. One advantage of electroporation is that large pieces of DNA, including artificial chromosomes, can be transformed by this method.

Ballistic transformation typically comprises the steps of: (a) providing a plant material as a target; (b) propelling a microprojectile carrying the heterologous nucleotide sequence at the plant target at a velocity sufficient to pierce the walls of the cells within the target and to deposit the nucleotide sequence within a cell of the target to thereby provide a transformed target. The method can further include the step of culturing the transformed target with a selection agent and, optionally, regeneration of a transformed plant. As noted below, the technique may be carried out with the nucleotide sequence as a precipitate (wet or freeze-dried) alone, in place of the aqueous solution containing the nucleotide sequence.

Any ballistic cell transformation apparatus can be used in practicing the present invention. Exemplary apparatus are disclosed by Sandford et al. (Particulate Science and Technology 5, 27 (1988)), Klein et al. (Nature 327, 70 (1987)), and in EP 0 270 356. Such apparatus have been used to transform maize cells (Klein et al., Proc. Natl. Acad. Sci. USA 85, 4305 (1988)), soybean callus (Christou et al., Plant Physiol. 87, 671 (1988)), McCabe et al., BioTechnology 6, 923 (1988), yeast mitochondria (Johnston et al., Science 240, 1538 (1988)), and Chlamydomonas chloroplasts (Boynton et al., Science 240, 1534 (1988)).

Alternately, an apparatus configured as described by Klein et al. (Nature 70, 327 (1987)) may be utilized. This apparatus comprises a bombardment chamber, which is divided into two separate compartments by an adjustable-height stopping plate. An acceleration tube is mounted on top of the bombardment chamber. A macroprojectile is propelled down the acceleration tube at the stopping plate by a gunpowder charge. The stopping plate has a borehole formed therein, which is smaller in diameter than the microprojectile. The macroprojectile carries the microprojectile(s), and the macroprojectile is aimed and fired at the borehole. When the macroprojectile is stopped by the stopping plate, the microprojectile(s) is propelled through the borehole. The target is positioned in the bombardment chamber so that a microprojectile(s) propelled through the bore hole penetrates the cell walls of the cells in the target and deposit the polynucleotide sequence of interest carried thereon in the cells of the target. The bombardment chamber is partially evacuated prior to use to prevent atmospheric drag from unduly slowing the microprojectiles. The chamber is only partially evacuated so that the target tissue is not desiccated during bombardment. A vacuum of between about 400 to about 800 millimeters of mercury is suitable.

In alternate embodiments, ballistic transformation is achieved without use of microprojectiles. For example, an aqueous solution containing the polynucleotide of interest as a precipitate may be carried by the macroprojectile (e.g., by placing the aqueous solution directly on the plate-contact end of the macroprojectile without a microprojectile, where it is held by surface tension), and the solution alone propelled at the plant tissue target (e.g., by propelling the macroprojectile down the acceleration tube in the same manner as described above). Other approaches include placing the nucleic acid precipitate itself (“wet” precipitate) or a freeze-dried nucleotide precipitate directly on the plate-contact end of the macroprojectile without a microprojectile. In the absence of a microprojectile, it is believed that the nucleotide sequence must either be propelled at the tissue target at a greater velocity than that needed if carried by a microprojectile, or the nucleotide sequenced caused to travel a shorter distance to the target (or both).

It particular embodiments, the nucleotide sequence is delivered by a microprojectile. The microprojectile can be formed from any material having sufficient density and cohesiveness to be propelled through the cell wall, given the particle's velocity and the distance the particle must travel. Non-limiting examples of materials for making microprojectiles include metal, glass, silica, ice, polyethylene, polypropylene, polycarbonate, and carbon compounds (e.g., graphite, diamond). Non-limiting examples of suitable metals include tungsten, gold, and iridium. The particles should be of a size sufficiently small to avoid excessive disruption of the cells they contact in the target tissue, and sufficiently large to provide the inertia required to penetrate to the cell of interest in the target tissue. Particles ranging in diameter from about one-half micrometer to about three micrometers are suitable. Particles need not be spherical, as surface irregularities on the particles may enhance their carrying capacity.

The nucleotide sequence may be immobilized on the particle by precipitation. The precise precipitation parameters employed will vary depending upon factors such as the particle acceleration procedure employed, as is known in the art. The carrier particles may optionally be coated with an encapsulating agents such as polylysine to improve the stability of nucleotide sequences immobilized thereon, as discussed in EP 0 270 356 (column 8).

Alternatively, plants may be transformed using Agrobacterium tumefaciens or Agrobacterium rhizogenes. Agrobacterium-mediated nucleic acid transfer exploits the natural ability of A. tumefaciens and A. rhizogenes to transfer DNA into plant chromosomes. Agrobacterium is a plant pathogen that transfers a set of genes encoded in a region called T-DNA of the Ti and Ri plasmids of A. tumefaciens and A. rhizogenes, respectively, into plant cells. The typical result of transfer of the Ti plasmid is a tumorous growth called a crown gall in which the T-DNA is stably integrated into a host chromosome. Integration of the Ri plasmid into the host chromosomal DNA results in a condition known as “hairy root disease”. The ability to cause disease in the host plant can be removed by deletion of the genes in the T-DNA without loss of DNA transfer and integration. The DNA to be transferred is attached to border sequences that define the end points of an integrated T-DNA.

Transfer by means of engineered Agrobacterium strains has become routine for many dicotyledonous plants. Some difficulty has been experienced, however, in using Agrobacterium to transform monocotyledonous plants, in particular, cereal plants. However, Agrobacterium mediated transformation has been achieved in several monocot species, including cereal species such as rye, maize (Rhodes et al., Science 240, 204 (1988)), and rice (Hiei et al., (1994) Plant J. 6:271).

While the following discussion will focus on using A. tumefaciens to achieve gene transfer in plants, those skilled in the art will appreciate that this discussion also applies to A. rhizogenes. Transformation using A. rhizogenes has developed analogously to that of A. tumefaciens and has been successfully utilized to transform, for example, alfalfa, Solanum nigrm L., and poplar (U.S. Pat. No. 5,777,200 to Ryals et al.). As described by U.S. Pat. No. 5, 773,693 to Burgess et al., it is preferable to use a disarmed A. tumefaciens strain (as described below), however, the wild-type A. rhizogenes may be employed. An illustrative strain of A. rhizogenes is strain 15834.

In particular protocols, the Agrobacterium strain is modified to contain the nucleotide sequences to be transferred to the plant. The nucleotide sequence to be transferred is incorporated into the T-region and is typically flanked by at least one T-DNA border sequence, optionally two T-DNA border sequences. A variety of Agrobacterium strains are known in the art particularly, and can be used in the methods of the invention. See, e.g., Hooykaas, Plant Mol. Biol. 13, 327 (1989); Smith et al., Crop Science 35, 301 (1995); Chilton, Proc. Natl. Acad, Sci. USA 90, 3119 (1993); Mollony et al., Monograph Theor. Appl. Genet NY 19, 148 (1993); Ishida et al., Nature Biotechnol. 14, 745 (1996); and Komari et al., The Plant Journal 10, 165 (1996).

In addition to the T-region, the Ti (or Ri) plasmid contains a vir region. The vir region is important for efficient transformation, and appears to be species-specific.

Two exemplary classes of recombinant Ti and Ri plasmid vector systems are commonly used in the art. In one class, called “cointegrate,” the shuttle vector containing the gene of interest is inserted by genetic recombination into a non-oncogenic Ti plasmid that contains both the cis-acting and trans-acting elements required for plant transformation as, for example, in the PMLJ1 shuttle vector of DeBlock et al., EMBO J 3, 1681 (1984), and the non-oncogenic Ti plasmid pGV2850 described by Zambryski et al., EMBO J 2, 2143 (1983). In the second class or “binary” system, the gene of interest is inserted into a shuttle vector containing the cis-acting elements required for plant transformation. The other necessary functions are provided in trans by the non-oncogenic Ti plasmid as exemplified by the pBIN19 shuttle vector described by Bevan, Nucleic Acids Research 12, 8711 (1984), and the non-oncogenic Ti plasmid PAL4404 described by Hoekma, et al., Nature 303, 179 (1983).

Binary vector systems have been developed where the manipulated disarmed T-DNA carrying the heterologous polynucleotide of interest and the vir functions are present on separate plasmids. In this manner, a modified T-DNA region comprising foreign DNA (the nucleic acid to be transferred) is constructed in a small plasmid that replicates in E. coli. This plasmid is transferred conjugatively in a tri-parental mating or via electroporation into A. tumefaciens that contains a compatible plasmid with virulence gene sequences. The vir functions are supplied in trans to transfer the T-DNA into the plant genome. Such binary vectors are useful in the practice of the present invention.

In particular embodiments of the invention, super-binary vectors are employed. See, e.g., U.S. Pat. No. 5,591,615 and EP 0 604 662. Such a super-binary vector has been constructed containing a DNA region originating from the hypervirulence region of the Ti plasmid pTiBo542 (Jin et al., J. Bacteriol. 169, 4417 (1987)) contained in a super-virulent A. tumefaciens A281 exhibiting extremely high transformation efficiency (Hood et al., Biotechnol. 2, 702 (1984); Hood et al., J. Bacteriol. 168, 1283 (1986); Komari et al., J. Bacteriol. 166, 88 (1986); Jin et al., J. Bacteriol. 169, 4417 (1987); Komari, Plant Science 60, 223 (1987); ATCC Accession No. 37394.

Exemplary super-binary vectors known to those skilled in the art include pTOK162 (Japanese patent Appl. (Kokai) No. 4-222527, EP 504,869, EP 604,662, and U.S. Pat. No. 5,591,616) and pTOK233 (Komari, Plant Cell Reports 9, 303 (1990); Ishida et al., Nature Biotechnology 14, 745 (1996)). Other super-binary vectors may be constructed by the methods set forth in the above references. Super-binary vector pTOK162 is capable of replication in both E. coli and in A. tumefaciens. Additionally, the vector contains the virB, virC and virG genes from the virulence region of pTiBo542. The plasmid also contains an antibiotic resistance gene, a selectable marker gene, and the nucleic acid of interest to be transformed into the plant. The nucleic acid to be inserted into the plant genome is typically located between the two border sequences of the T region. Super-binary vectors of the invention can be constructed having the features described above for pTOK162. The T-region of the super-binary vectors and other vectors for use in the invention are constructed to have restriction sites for the insertion of the genes to be delivered. Alternatively, the DNA to be transformed can be inserted in the T-DNA region of the vector by utilizing in vivo homologous recombination. See, Herrera-Esterella et al., EMBO J. 2, 987 (1983); Horch et al., Science 223, 496 (1984). Such homologous recombination relies on the fact that the super-binary vector has a region homologous with a region of pBR322 or other similar plasmids. Thus, when the two plasmids are brought together, a desired gene is inserted into the super-binary vector by genetic recombination via the homologous regions.

In plants stably transformed by Agrobacteria-mediated transformation, the polynucleotide of interest is incorporated into the plant nuclear genome, typically flanked by at least one T-DNA border sequence and generally two T-DNA border sequences.

Plant cells may be transformed with Agrobacteria by any means known in the art, e.g., by co-cultivation with cultured isolated protoplasts, or transformation of intact cells or tissues. The first uses an established culture system that allows for culturing protoplasts and subsequent plant regeneration from cultured protoplasts. Identification of transformed cells or plants is generally accomplished by including a selectable marker in the transforming vector, or by obtaining evidence of successful bacterial infection.

Methods of introducing a nucleic acid into a plant can also comprise in vivo modification of genetic material, methods for which are known in the art. For example, in vivo modification can be used to insert a nucleic acid comprising a promoter sequence of the invention into the plant genome.

Suitable methods for in vivo modification include the techniques described in Gao et. al., Plant J. 61, 176 (2010); Li et al., Nucleic Acids Res. 39, 359 (2011); U.S. Pat. Nos. 7,897,372 and 8,021,867; U.S. Patent Publication No. 2011/0145940 and in International Patent Publication Nos. WO 2009/114321, WO 2009/134714 and WO 2010/079430. For example, one or more transcription affector-like nucleases (TALEN) and/or one or more meganucleases may be used to incorporate an isolated nucleic acid comprising a promoter sequence of the invention into the plant genome. In representative embodiments, the method comprises cleaving the plant genome at a target site with a TALEN and/or a meganuclease and providing a nucleic acid that is homologous to at least a portion of the target site and further comprises a nucleotide sequence of this invention (e.g., SEQ ID NOs:1-20), such that homologous recombination occurs and results in the insertion of the nucleotide sequence of the invention into the genome.

Protoplasts, which have been transformed by any method known in the art, can also be regenerated to produce intact plants using known techniques.

Plant regeneration from cultured protoplasts is described in Evans et al., Handbook of Plant Cell Cultures, Vol. 1: (MacMilan Publishing Co. New York, 1983); and Vasil I. R. (ed.), Cell Culture and Somatic Cell Genetics of Plants, Acad. Press, Orlando, Vol. I, 1984, and Vol. II, 1986). Essentially all plant species can be regenerated from cultured cells or tissues, including but not limited to, all major species of sugar-cane, sugar beet, cotton, fruit trees, and legumes.

Means for regeneration vary from species to species of plants, but generally a suspension of transformed protoplasts or a petri plate containing transformed explants is first provided. Callus tissue is formed and shoots may be induced from callus and subsequently root. Alternatively, somatic embryo formation can be induced in the callus tissue. These somatic embryos germinate as natural embryos to form plants. The culture media will generally contain various amino acids and plant hormones, such as auxin and cytokinins. It is also advantageous to add glutamic acid and proline to the medium, especially for such species as corn and alfalfa. Efficient regeneration will depend on the medium, on the genotype, and on the history of the culture. If these three variables are controlled, then regeneration is usually reproducible and repeatable.

The regenerated plants are transferred to standard soil conditions and cultivated in a conventional manner. The plants are grown and harvested using conventional procedures.

Alternatively, transgenic plants may be produced using the floral dip method (See, e.g., Clough and Bent (1998) Plant Journal 16:735-743, which avoids the need for plant tissue culture or regeneration. In one representative protocol, plants are grown in soil until the primary inflorescence is about 10 cm tall. The primary inflorescence is cut to induce the emergence of multiple secondary inflorescences. The inflorescences of these plants are typically dipped in a suspension of Agrobacterium containing the vector of interest, a simple sugar (e.g., sucrose) and surfactant. After the dipping process, the plants are grown to maturity and the seeds are harvested. Transgenic seeds from these treated plants can be selected by germination under selective pressure (e.g., using the chemical bialaphos). Transgenic plants containing the selectable marker survive treatment and can be transplanted to individual pots for subsequent analysis. See Bechtold, N. and Pelletier, G. Methods Mol Biol 82, 259-266 (1998); Chung, M. H. et al. Transgenic Res 9, 471-476 (2000); Clough, S. J. and Bent, A. F. Plant J 16, 735-743 (1998); Mysore, K. S. et al. Plant J 21, 9-16 (2000); Tague, B. W. Transgenic Res 10, 259-267 (2001); Wang, W. C. et al. Plant Cell Rep 22, 274-281 (2003); Ye, G. N. et al. Plant J., 19:249-257 (1999).

The particular conditions for transformation, selection and regeneration can be optimized by those of skill in the art. Factors that affect the efficiency of transformation include the species of plant, the target tissue or cell, composition of the culture media, selectable marker genes, kinds of vectors, and light/dark conditions. Therefore, these and other factors may be varied to determine an optimal transformation protocol for any particular plant species. It is recognized that not every species will react in the same manner to the transformation conditions and may require a slightly different modification of the protocols disclosed herein. However, by altering each of the variables, an optimum protocol can be derived for any plant species.

Further, the genetic properties engineered into the transgenic seeds and plants, plant parts, and/or plant cells of the present invention described herein can be passed on by sexual reproduction or vegetative growth and therefore can be maintained and propagated in progeny plants. Generally, maintenance and propagation make use of known agricultural methods developed to fit specific purposes such as harvesting, sowing or tilling.

The present invention is more particularly described in the following examples that are intended as illustrative only since numerous modifications and variations therein will be apparent to those skilled in the art.

### EXAMPLES

### Example 1

### The Early Transcriptome Response to Drought and Rehydration by Root Hairs of Maize (Zea mays L.)

Transcriptome studies involving water stress have primarily been conducted using whole plants or intact organs including intact root systems [10, 13-18]. However, the tissue in direct contact with water is the root epidermis. Significantly contributing to the root epidermal surface area are root hairs (RH), single cell epidermal projections that mediate nutrient uptake, water uptake and interaction with root symbionts [19-21].

Physiologically, RH have been shown to respond to drought or osmotic stress by reducing tip elongation and becoming swollen [23-26] in a process likely involving ABA [24]. It is well known that RH elongation is mediated by numerous interacting signalling pathways that cause polarized tip growth [27]. Laboratory experiments suggest that when rainfall resumes following water limitation, water and its dissolved nutrients can rehydrate individual RH which in turn then act as conduits for water for the whole plant [19]. Only a portion of the RH surface area appears to uptake water, with young RH taking up large quantities of water compared to older RH [19]. In the absence of tolerance mechanisms, damage to RH caused by drought would affect the long-term ability of a plant to interact with its environment [21].

As noted above, RH comprise the majority of the plant root surface area for water absorption. Nevertheless, no studies have reported the genome-wide transcriptome response of RH to changes in water availability in any species. Here we examined the early transcriptome response of maize RH to rehydration following an extended drought. We also searched for over-represented putative cis-acting motifs present in the promoters of the genes that were differentially expressed. Our results suggest two classes of drought-tolerance genes acting in RH, a late recovery group that continued to be up-regulated following watering, and a down-regulated early recovery class, each under the control of distinct candidate cis-acting motifs. In this study, an Affymetrix-46K microarray was used to examine the RH transcriptome response to rehydration following drought using maize/corn (Zea mays L.). At least 1831 annotated genes were differentially expressed in maize RH during the first 3 h of rehydration after drought.

Seedlings were grown in slant tubes half-filled with nutrient agar to maximize the subsequent RH harvest. Tubes were placed in a custom growth system to reduce light exposure to roots. After germination, approximately half of the RH grew in air but with 1 ml of nutrient water added daily to prevent complete desiccation, and hence were exposed to mild drought stress. The remaining RH grew in agar. At 10 days after germination, the roots were fully rehydrated using an aqueous solution that contained the same concentrations of nutrients present in the agar. RH were then harvested at 30 min and 3 h after rehydration for microarray analysis. In order to analyze the results, gene an annotations from MapMan and MaizeGenome.org were used in this study. A total of 29110 gene annotations were successfully retrieved from the 46K microarray using a custom Peri script. Array expression patterns were grouped using Principal Component Analysis. The samples before and after rehydration were clearly separated indicating a genetic response to the treatment. A total of 1645 probes representing 1831 genes were differentially expressed between time 0, 30 min and 3 h after rehydration. Approximately half of the these genes showed altered regulation within 30 min (t30 min, 30 min minus time 0 comparison) while the other half responded within 3 h (t3 h, 3 h minus time 0 comparison). No genes appeared to be differentially expressed between 3 h versus 30 min after rehydration, perhaps because this variation was much lower than what was observed at t30 min or t3 h, masking it by the statistical model used (see Methods).

### Prediction of Cis-Acting Promoter Motifs

A search was performed for cis-acting motifs in the promoters (−1000 to +1) of the genes that were differentially expressed in maize RH following rehydration. Promoters were first grouped into non-ambiguous gene expression clusters where possible. Shared de novo motifs within each cluster were then searched [157], but only the most over-represented motifs are presented here. The majority of the candidate motifs were previously identified as regulating the transcription of seed storage protein genes, many of which are regulated by ABA [158, 159].

First, two seed storage protein promoter motifs were retrieved in Cluster 1 which included 839 genes highly expressed during drought but then repressed within 30 min following rehydration. The first promoter motif over-represented in Cluster 1 was SEF3, first identified as a binding site for Soybean Embryo Factor 3, responsible for transcription of seed storage protein β-conglycinin [160, 161]. The second Cluster 1 promoter motif retrieved (CAAACNCAC) was highly similar to the CA(n)-element CAAACAC first identified as part of the B-box in the promoter of the gene encoding Brassica napus seed storage protein napin A (napA) [162, 163]. The CA(n)-element was shown to be one of two motifs required for seed specific expression, the other being the ABA Response Element, ABRE [3, 163]. The CA(n)-element was shown to be conserved in >103 out of 113 seed storage protein promoters across species and thought to amplify the response of the ABRE box [163]. A napin A ortholog was recently shown to be induced during drought in maize plants [18].

The 583 genes in Cluster 2 were repressed during drought but activated by watering, achieving peak expression within 30 min. Cluster 2 motifs included CCTGTTC, highly similar to a motif previously identified in the promoter of the rice seed storage protein glutenin gene [164]. Two closely related motifs highly similar to the bZIP Vascular Specificity Factor (VSF-1) binding site GCTCCGTTG [165] were also over-represented in Cluster 2 promoters. The VSF-1 binding site has been found in promoters of maize seed storage protein zein genes [166] as well as the promoter of the ABA-inducible LEA gene AtEm1 [167].

Two additional clusters, not originally separated from Clusters 1 and 2 using the microarray empirical Bayesian model [168], were identified using the HOPACH clustering method [169]. The 259 genes in Cluster 3 were repressed during drought and reactivated by water, achieving peak expression 3 h later. Three motifs closely related to a motif over-represented in anaerobically-induced genes (ANAERO4CONSENSUS, GTTTHGCAA) [170] were abundant in Cluster 3 promoters. One possibility is that RH experienced mild anoxia 3 h after the water treatment in spite of the presence of an oxygen source, as noted above. Another two motifs retrieved in Cluster 3 promoters contained the core consensus sequence CCCGCTT, highly related to the BS1 EgCCR gene promoter motif (CCCGCT). This motif was originally identified in the promoter of the Eucalyptus gunnii cinnamoyl-CoA reductase gene, which encodes the first step in lignin biosynthesis [171]. The BS1 EgCCR motif was located adjacent to MYB binding sites, but was not required for MYB binding [172]. More recently, the BS1 EgCCR motif was found in four seed-specific promoters in maize, but whether or not this motif contributes to this tissue specificity has not been reported [173].

Cluster 4 was intriguing as these genes were highly expressed during drought, repressed within 30 min following rehydration, but then moderately reactivated 2.5 hours later. The RY-repeat motif, also known as the FUS3/Sph motif (CATGCA)[158, 174], was present in the promoters of 17 out the 18 genes in Cluster 4. The RY motif can bind Domain 3 of the master ABA transcription factors ABI3 and VP1, and is thought to be the ancestral target of these proteins, now replaced by ABRE in angiosperms)[158, 174, 175]. The RY motif controls late embryogenesis and seed development, and its ACGT core regulates cereal seed storage genes [174, 176]. The RY motif may also help to mediate signal cross-talk, as the B3 domain is present in proteins related to ABA, ethylene (ERF) and auxin (ARF) signalling as well as the cold responsive transcription factor RAV1 [177, 178]. At least two genes encoding B3 domain-containing RAV proteins (PF02362), ZmRAV1 (GRMZM2G169654) and a RAV (GRMZM2G018336), were differentially expressed in maize RH in response to rehydration.

Most of the Cluster 4 promoters also contained a Squamosa Promoter Binding Protein (SBP)-box, shown to be involved primarily in floral development but also trichome initiation [73]. This result is predictive of a jasmonate −Tasselseed 1 (Ts1) regulon acting in RH through miR156, as the latter two regulators have been implicated in regulating a subset of genes in maize containing an SBP box [72, 73].

Many of the above cis-acting motifs and their binding proteins have been described as being completely seed specific [159]. Indeed some of the motifs identified here are often found adjacent to one another in the promoters of genes encoding seed storage molecules. For example, the upstream sequence of the soybean oleosin gene contains SEF, CA(n), RY and ABRE elements [179]. Without being limited to a particular theory, the results suggest that the regulatory programs operating in seeds, including those involving ABA signaling, may also be functioning in RH to regulate drought/rehydration responses.

### Methods

### Biological Material

Maize inbred line B73 originated from the USDA Stock Center (PI 550473, North Central Regional PI Station, Ames, Iowa) was used for this study.

### Growth System Overview

In the optimized RH growth system, 30 ml of root hair media (RHM) agar (see below) was added into each sterile glass test tube (25×200 mm, Pyrex® Vista™ culture tube, Sigma-Aldrich, St Louis, USA) which was capped with a rubber stopper (Black Rubber Stoppers, #59580-182, VWR, USA). The agar was left to solidify at a 90° -horizontal slant. The RHM agar contained nitrate and was adapted from previous studies [180, 181]; it consisted of 3% (w/v) agar (Sigma-Aldrich A1296), 0.15 mM Ca(NO₃)₂ (Sigma Z37124), 4.85 mM CaCl₂ (Sigma 10043-52-4), 0.2 mM K₂SO₄ (Sigma 77778-80-5), 0.82 mM MgSO₄ (Sigma 10034-998), 0.3 mM CaNO₃ (Sigma Z37124), 0.1 mM FeNa-EDTA (PhytoTechnology Laboratories 15708-41-5) 9.1 μM MgCl₂ (Fisher Biotech 7773-61-5), 0.034 μM NaMoO₄ (Sigma 10102-40-6), 18 μM H₃B0₃ (EMD BX0865-1), 0.08 mM Ca₅OH(PO₄)₃ (Fluka 21218), 0.2 μM CuSO₄ (Sigma C7631), and 0.4 μM ZnSO₄ (Sigma 7446-20-0), pH 5.5-6.0. A single germinated seed was placed onto the agar surface, 10-20 mm from the top of the tube. A home-made foam plug was used to gently press the seed against the agar, which was covered by pieces of rockwool; this system maintained the seed at the correct position, and prevented dessication, pathogen contamination and light exposure. The RHM agar contained calcium phosphate tri-basic to simulate RH growth [180, 181]. Roots grew along the agar surface: half of the RH penetrated the agar, which allowed for a uniform exchange of nutrients and mechanical resistance, while the other half of the RH grew into the air which facilitated gas exchange. Since the RH grew in the air and in soft agar, harvesting caused minimal RH damage. The tubes were placed into adapted growth boxes which were then placed into growth chambers.

### Growth and Rehydration Treatments

Seeds were germinated on water-saturated Whatmann paper, and grown in the dark at about 28° C. Uniformly germinated 2-3 day old seedlings were transferred to the RHM agar test tubes (see above). The growth conditions consisted of a 16 h photoperiod, 30° C. day/22° C. night, with 250-300 μMol m-² s⁻¹ light (incandescent bulbs, and full spectrum fluorescent light incandescent light bulbs). Plants were watered every day with about 1 ml of RHM solution to prevent complete desiccation of the RH growing in air, and hence the air-exposed RH experienced a mild localized drought. At 8 days after transfer (about 10 days after germination: time 0), liquid RHM containing 3% (v/v) H₂O₂ as an oxygen source was added to the tubes. Roots were harvested and frozen in liquid nitrogen at three time points: time 0, 30 min and 3 h. There were 8-12 plants/time point/replicate and the experiment was replicated three times in different growth chambers.

### Root Hair RNA Extraction

Long forceps were used to pull intact roots attached to agar from the tubes. Root segments below the most apical lateral root node were dissected and frozen in liquid nitrogen (LiqN) individually in 1.5 ml Eppendorf tubes which were then stored at −80° C. For RNA extraction, tubes were placed in LiqN. One by one, LiqN was poured into each tube; a tweezer pre-dipped in LiqN was used to remove the root and place it above a clean Eppendorf tube containing 200 μTriReagent (AM9738, Ambion, USA). A second pre-chilled tweezer was then used to shave the RH; the RH remained attached to the tweezer which was then dipped into the TriReagent. One TriReagent tube was used to harvest RH RNA for every 2-3 plants. All tubes were re-frozen at −80° C. so that all RNA per replicate could be extracted simultaneously. TriReagent samples from each replicate/time point were pooled to a maximum of 800 μl which was topped up to 1 ml with TriReagent. Samples were vortexed for 30 s, incubated at room temperature (RT) for 4-6 min, then centrifuged at 12000×g for 10 min at 4° C. The aqueous phase was removed to a new tube, which was extracted with 200 μl chloroform:isoamyl alcohol (24:1) by vortexing for 15 s and incubating at RT for 3 min. Samples were centrifuged at 12000×g for 10 min at 4° C. The upper phase was transferred to new tube to which was added 250 μl isopropanol (RT) and 250 μl of high salt solution (0.8 M sodium citrate and 1.2 M sodium chloride) in order to eliminate polysaccharides and cell wall waste [182]. The samples were incubated for 15 min at RT, and then centrifuged at 12000×g for 10 min at 4° C. The supernatant was carefully removed and discarded; the RNA-containing pellet was translucent and was resuspended in 500 μl RNAse-free water. Next, 400 μl of Acid-Phenol:Chloroform (AM9720, Ambion, USA) was added to each tube; samples were vortexed for 10 s, incubated for 3 min at RT, then centrifuged at 12000×g for 10 min at 4° C. The upper phase was transferred to a new tube, to which was added 500 μl isopropanol and 66 μl 3M sodium acetate pH 5.2. The samples were vortexed briefly, incubated on ice for 10 min, then 12000×g for 10 min at 4° C. The supernatant was discarded, and the pellet washed twice with 1 ml 75% ethanol with 5 min centrifugations at 12000×g at 4° C. The pellets were slightly dried and the RNA dissolved in 50 μl of RNAse-free water with a 10 min incubation at 55° C. and periodic vortexing.

### Microarray Hybridization, Analysis and Clustering

The custom Syngenta B73 Corn 46K Affymetrix array was used to hybridize RH RNA as previously described [183]. RNA from the three time points (time 0, 30 min, 3 h) were hybridized in biological triplicates. Bioconductor [184] and R [185] were used for subsequent gene expression analysis. Expression normalization was performed using the RMA method [186],

Differential gene expression was measured using a linear model from the Limma Package [187]. The linear model was adjusted using the empirical Bayesian method [187]. To define the effect of nitrogen at each time point after the treatment shift, gene expression was compared each reciprocal comparison time 0, 30 min and 3 h after rehydration. For each comparison, the p-value of the Empirical Bayesian test was corrected using the Benjamini-Hochberg method [188]. The p-value was set at 0.05. Gene annotations were retrieved using MapMan (Zm_B73 file, accessed April 11) [189] and MaizeSequence.org [190] as starting points to link genes to the corresponding microarray probes and annotation category via a custom Peri script. The initial Mapman annotation file had 29,142 genes, but could only identify 34% of the probes on the microarray. After the use of MaizeSequence.org annotations, gene annotations could be retrieved for 65% of probes. Annotation categories that were overrepresented were identified using Fisher's exact test using R programming.

### Promoter Motif Prediction

The 1831 differentially expressed genes were clustered using the HOPACH method [169]. Each of the four gene expression clusters were searched for shared promoter motifs. De-novo cis-acting motifs (≧6 bp) in the −1000 to +1 promoter region were predicted using a Perl-based motif discovery program that was custom developed for the maize genome, called Promzea [157]. Gene promoters were identified by taking 1000 by upstream of their Gramene ID sequences (longest cDNA) in the MaizeSequence.org database. Over-represented motifs in each promoter were identified using three motif discovery tools: Weeder [191], MEME [192] and BioProspector [193]. Each motif was re-evaluated using one of the following statistical methods: the hypergeometric test or the binomial test. All significant motifs found in the search were compared to the motif databases, AGRIS [194], Athamap [195] and PLACE [196], using STAMP software [197]. In order to reduce false positives and to increase motif discovery accuracy, only the best 100 promoters from Clusters 1,2 and 3 were selected for motif discovery based on their adjusted p-values (Benjamini-Hochberg as noted above). Cluster 4 had <100 promoters.

### REFERENCES

10. Oono Y, Seki M, Nanjo T, Narusaka M, Fujita M, Satoh R, Satou M, Sakurai T, Ishida J, Akiyama K et al: Monitoring expression profiles of Arabidopsis gene expression during rehydration process after dehydration using ca. 7000 full-length cDNA microarray. The Plant Journal 2003, 34(6):868-887.
13. Wang D, Pan Y, Zhao X, Zhu L, Fu B, Li Z: Genome-wide temporal-spatial gene expression profiling of drought responsiveness in rice. BMC Genomics 2011, 12(1):149.
14. Rodriguez M C S, Edsgärd D, Hussain S S, Alquezar D, Rasmussen M, Gilbert T, Nielsen B H, Bartels D, Mundy J: Transcriptomes of the desiccation-tolerant resurrection plant Craterostigma plantagineum. The Plant Journal 2010, 63(2):212-228.
15. Rabbani M A, Maruyama K, Abe H, Khan M A, Katsura K, Ito Y, Yoshiwara K, Seki M, Shinozaki K, Yamaguchi-Shinozaki K: Monitoring Expression Profiles of Rice Genes under Cold, Drought, and High-Salinity Stresses and Abscisic Acid Application Using cDNA Microarray and RNA Gel-Blot Analyses. Plant Physiology 2003, 133(4):1755-1767.
16. Lee T-H, Kim Y-K, Pham T T M, Song S I, Kim J-K, Kang K Y, An G, Jung K-H, Galbraith D W, Kim M et al: RiceArrayNet: A Database for Correlating Gene Expression from Transcriptome Profiling, and Its Application to the Analysis of Coexpressed Genes in Rice. Plant Physiology 2009, 151(1):16-33.
17. Marino R, Ponnaiah M, Krajewski P, Frova C, Gianfranceschi L, Pè M, Sari-Gorla M: Addressing drought tolerance in maize by transcriptional profiling and mapping. Molecular Genetics and Genomics 2009, 281(2):163-179.
18. Zheng J, Fu J, Gou M, Huai J, Liu Y, Jian M, Huang Q, Guo X, Dong Z, Wang H et al: Genome-wide transcriptome analysis of two maize inbred lines under drought stress. Plant Molecular Biology 2010, 72(4):407-421.
19. Cailloux M: Metabolism and the absorption of water by root hairs. Canadian Journal of Botany 1972, 50:557-573.
20. Peterson R, Farquhar M: Root hairs: Specialized tubular cells extending root surfaces. The Botanical Review 1996, 62(1):1-40.
21. Allen M F: Linking water and nutrients through the vadose zone: a fungal interface between the soil and plant systems. Journal of Arid Land 2011, 3(3):155-163.
23. Zahran H H, Sprent J I: Effects of sodium chloride and polyethylene glycol on root-hair infection and nodulation of Vicia faba L. plants by Rhizobium leguminosarum. Planta 1986, 167(3):303-309.
24. Schnall J A, Quatrano R S: Abscisic Acid Elicits the Water-Stress Response in Root Hairs of Arabidopsis thaliana. Plant Physiology 1992, 100(1):216-218.
25. Dauphin A, El-Maarouf H, Vienney N, Rona J-P, Bouteau F: Effect of desiccation on potassium and anion currents from young root hairs: Implication on tip growth. Physiologia Plantarum 2001, 113(1):79-84.
26, Volgger M, Lang I, Ove\check{c}ka M, Lichtscheidl I: Plasmolysis and cell wall deposition in wheat root hairs under osmotic stress. Protoplasma 2010, 243(1):51-62.
27. Libault M, Brechenmacher L, Cheng J, Xu D, Stacey G: Root hair systems biology. Trends in Plant Science 2010, 15(10:641-650.
72. Hultquist J, Dorweiler J: Feminized tassels of maize mop1 and ts1 mutants exhibit altered levels of miR156 and specific SBP-box genes. Planta 2008, 229(1):99-113.
73. Shikata M, Koyama T, Mitsuda N, Ohme-Takagi M: Arabidopsis SBP-Box Genes SPL10, SPL11 and SPL2 Control Morphological Change in Association with Shoot Maturation in the Reproductive Phase. Plant and Cell Physiology 2009, 50(12):2133-2145.
157. Liseron-Monfils C V, Ashlock D, McNicholas P D, Fauteux F, Strömvik M, Raizada M N: Promzea: A pipeline for discovery of regulatory motifs in maize (Zea mays L.) and its application to the anthocyanin biosynthetic pathway. 2011:submitted.
158. Giraudat J, Hauge B M, Valon C, Smalle J, Parcy F, Goodman H M: Isolation of the Arabidopsis ABI3 Gene by Positional Cloning. The Plant Cell Online 1992, 4(10):1251-1261.
159. Thomas T L: Gene Expression during Plant Embryogenesis and Germination: An Overview. The Plant Cell Online 1993, 5(10):1401-1410.
160. Allen R D, Bernier F, Lessard P A, Beachy R N: Nuclear Factors Interact with a Soybean [beta]-Conglycinin Enhancer. The Plant Cell Online 1989, 1(6):623-631.
161. Lessard P A, Allen R D, Bernier F, Crispino J D, Fujiwara T, Beachy R N: Multiple nuclear factors interact with upstream sequences of differentially regulated β-conglycinin genes. Plant Molecular Biology 1991, 16(3):397-413.
162. Ezcurra I, Wycliffe P, Nehlin L, Ellerström M, Rask L: Transactivation of the Brassica napus napin promoter by ABI3 requires interaction of the conserved B2 and B3 domains of ABI3 with different cis-elements: B2 mediates activation through an ABRE, whereas B3 interacts with an RY/G-box. The Plant Journal 2000, 24(1):57-66.
163. Stålberg K, Ellerstöm M, Ezcurra I, Ablov S, Rask L: Disruption of an overlapping E-box/ABRE motif abolished high transcription of the napA storage-protein promoter in transgenic Brassica napus seeds. Planta 1996, 199(4):515-519.
164. Kim S Y, Wu R: Multiple protein factors bind to a rice glutelin promoter region. Nucleic Acids Research 1990, 18(23):6845-6852.
165. Ringli C, Keller B: Specific interaction of the tomato bZIP transcription factor VSF-1 with a non-palindromic DNA sequence that controls vascular gene expression. Plant Molecular Biology 1998, 37(6):977-988.
166. So J-S, Larkins BA: Binding of an endosperm-specific nuclear protein to a maize beta-zein gene correlates with zein transcriptional activity. Plant Molecular Biology 1991, 17(3):309-319.
167. Vicient C M, Hull G, Guilleminot J, Devic M, Delseny M: Differential expression of the Arabidopsis genes coding for Em-like proteins. Journal of Experimental Botany 2000, 51(348):1211-1220.
168. Smyth G K: Linear models and empirical bayes methods for assessing differential expression in microarray experiments. Statistical Applications in Genetics and Molecular Biology 2004, 3(1):Article3.
169. Van der Laan M J, Pollard K S: A new algorithm for hybrid hierarchical clustering with visualization and the bootstrap. Journal of Statistical Planning and Inference 2003, 117(2):275-303.
170. Mohanty B, Krishnan S P T, Swarup S, Bajic V B: Detection and Preliminary Analysis of Motifs in Promoters of Anaerobically Induced Genes of Different Plant Species. Annals of Botany 2005, 96(4):669-681.
171. Lacombe E, Van Doorsselaere J, Boerjan W, Boudet A M, Grima-Pettenati J: Characterization of cis-elements required for vascular expression of the Cinnamoyl CoA Reductase gene and for protein—DNA complex formation. The Plant Journal 2000, 23(5):663-676.
172. Rahantamalala A, Rech P, Martinez Y, Chaubet-Gigot N, Grima-Pettenati J, Pacquit V: Coordinated transcriptional regulation of two key genes in the lignin branch pathway—CAD and CCR—is mediated through MYB-binding sites. BMC Plant Biology 2010, 10(1):130.
173. Fu H: Expression cassettes for embryo-specific expression in plants. In. Edited by GMBH BPSC. US; 2011.
174. Reidt W, Wohlfarth T, Ellerström M, Czihal A, Tewes A, Ezcurra I, Rask L, Bäumlein H: Gene regulation during late embryogenesis: the RY motif of maturation-specific gene promoters is a direct target of the FUS3 gene product. The Plant Journal 2000, 21(5):401-408.
175. Sakata Y, Nakamura I, Taji T, Tanaka S, Quatrano R S: Regulation of the ABA-responsive Em promoter by ABI3 in the moss Physcomitrella patens: Role of the ABA response element and the RY element. Plant Signaling & Behavior 2010, 5(9):1061-1066.
176. Yamamoto M P, Onodera Y, Touno S M, Takaiwa F: Synergism between RPBF Dof and RISBZ1 bZIP Activators in the Regulation of Rice Seed Expression Genes. Plant Physiology 2006, 141(4):1694-1707.
177. Nag R, Maity M, DasGupta M: Dual DNA Binding Property of ABA insensitive 3 Like Factors Targeted to Promoters Responsive to ABA and Auxin. Plant Molecular Biology 2005, 59(5):821-838.
178. Yamasaki K, Kigawa T, Inoue M, Tateno M, Yamasaki T, Yabuki T, Aoki M, Seki E, Matsuda T, Tomo Y et al: Solution Structure of the B3 DNA Binding Domain of the Arabidopsis Cold-Responsive Transcription Factor RAV 1. The Plant Cell Online 2004, 16(12):3448-3459.
179. Rowley D L, Herman E M: The upstream domain of soybean oleosin genes contains regulatory elements similar to those of legume storage proteins. Biochimica et Biophysica Acta (BBA)—Lipids and Lipid Metabolism 1997, 1345(1):1-4.
180. Liu G-D, Dunlop J, Phung T: Exotically long and prolific root hairs of Arabidopsis, corn and wheat grown in a phosphate nutrient buffer system. In: Plant Nutrition. 2001: 586-587.
181. Liu G-D, Dunlop J, Phung T: Induction of Root Hair Growth in a Phosphorus-Buffered Culture Solution. Agricultural Sciences in China 2006, 5(5):370-376.
182. Chomczynski P, Mackey K: Short technical reports. Modification of the TRI reagent procedure for isolation of RNA from polysaccharide- and proteoglycan-rich sources. BioTechniques 1995, 19(6):942-945.
183. Wagner F, Radelof U: Performance of different small sample RNA amplification techniques for hybridization on Affymetrix GeneChips. Journal of Biotechnology 2007, 129(4):628-634.
184. Gentleman R, Carey V, Bates D, Bolstad B, Dettling M, Dudoit S, Ellis B, Gautier L, Ge Y, Gentry J et al: Bioconductor: open software development for computational biology and bioinformatics. Genome Biology 2004, 5(10):R80.
185. R: A Language and Environment for Statistical Computing [www.R-project.org]
186. Irizarry R A, Bolstad B M, Collin F, Cope L M, Hobbs B, Speed T P: Summaries of Affymetrix GeneChip probe level data. Nucleic Acids Research 2003, 31(4):e15.
187. Smyth G: limma: Linear Models for Microarray Data. In: Bioinformatics and Computational Biology Solutions Using R and Bioconductor. Edited by Gentleman R, Carey V J, Huber W, Irizarry R A, Dudoit S: Springer New York; 2005: 397-420.
188. Benjamini Y, Hochberg Y: Controlling the False Discovery Rate: A Practical and Powerful Approach to Multiple Testing. Journal of the Royal Statistical Society Series B 1995, 57:289-300.
189. Thimm O, Biäsing O, Gibon Y, Nagel A, Meyer S, Krüger P, Selbig J, Müller L A, Rhee S Y, Stitt M: mapman: a user-driven tool to display genomics data sets onto diagrams of metabolic pathways and other biological processes. The Plant Journal 2004, 37(6):914-939.
190. Schnable P S, Ware D, Fulton R S, Stein J C, Wei F, Pasternak S, Liang C, Zhang J, Fulton L, Graves T A et al: The B73 maize genome: complexity, diversity, and dynamics. Science 2009, 326(5956):1112-1115.
191. Pavesi G, Zambelli F, Pesole G: Weeder H: an algorithm for finding conserved regulatory motifs and regions in homologous sequences. BMC Bioinformatics 2007, 8(1):46.
192. Bailey T L, Elkan C: Fitting a mixture model by expectation maximization to discover motifs in biopolymers. In: Proceedings of the Second International Conference on Intelligent Systems for Molecular Biology. Menlo Park, Calif.: AAAI Press; 1994: 28-36.
193. Liu X, Brutlag D, Liu J: BioProspector: discovering conserved DNA motifs in upstream regulatory regions of co-expressed genes. In: Pacific Symposium on Biocomputing 2001. Edited by Altman R B, Dunker A K, Hunter L, Klein T E; 2001: 127-138.
194. Yilmaz A, Mejia-Guerra M K, Kurz K, Liang X, Welch L, Grotewold E: AGRIS: the Arabidopsis Gene Regulatory Information Server, an update. Nucleic Acids Research 2011, 39(suppl 1):D1118-D1122.
195. Billow L, Engelmann S, Schindler M, Hehl R: AthaMap, integrating transcriptional and post-transcriptional data. Nucleic Acids Research 2009, 37(suppl 1):D983-D986.
196. Higo K, Ugawa Y, Iwamoto M, Korenaga T: Plant cis-acting regulatory DNA elements (PLACE) database: 1999. Nucleic Acids Research 1999, 27(1):297-300.
197. Mahony S, Benos P V: STAMP: a web tool for exploring DNA-binding motif similarities. Nucleic Acids Research 2007:253-258.

### Example 2

### Early Reprogramming of the Maize Root Hair Transcriptome in Response to Nitrate

The tissue in direct contact with soil nitrogen is the root epidermis. Root hairs (RH), which are single cell epidermal projections, comprise up to about 70% of the root epidermal surface area [6]. RH also interact with arbuscular mycorhizal fungi and soil microbes for enhanced nutrient uptake [7]. Though earlier reports have begun to characterize the RH transcriptome and proteome [8-10], no studies have been reported on the genome-wide transcriptome response of RH to soil nutrients including nitrogen in any plant species.

Recently, we reported that maize growing in aeroponics responds to optimal inorganic nitrogen (20 mM total N) by increasing both the length and number of RH compared to plants growing under limited nitrogen (8 mM total N) (both 3:1, NO₃⁻:NH₄⁺) [25]. These RH responses were determined to be an ancient trait as they were observed in the extant wild ancestor of maize, Balsas teosinte [26]. Enhanced RH growth by high nitrogen was consistent with studies in wheat in this concentration range [27]. In Arabidopsis, the combination of ammonium and nitrate also induced polar RH growth [28]. At lower nitrogen concentration ranges, NH₄⁺ and NO₃⁻ were shown to reduce RH growth or have no effect in other grasses and other dicots [29, 30]. All of these studies suggest that nitrogen can affect RH elongation, though possibly in a species or concentration dependent manner.

Here we examine the genome-wide transcriptome response of maize seedling RH at 30 min and 3 h after supplementation with added nitrate (AddN) compared to seedlings maintained in limiting, maintenance nitrate (MaintN). A challenge of conducting such studies is the need to change the nitrogen concentration without damaging RH and also being able to harvest sufficient RH-specific RNA for microarray analysis. These challenges were overcome here using a slant-tube agar/liquid root hair media (RHM) system combined with optimized RH RNA isolation procedures. Using these methodologies, we characterize the early reprogramming of the maize RH transcriptome in response to nitrate and identify candidate cis-acting promoter motifs underlying the distinct RH expression clusters observed.

The expression of >876 genes was significantly altered within 3 h of added nitrate in maize RH, including genes encoding 127 putative transcription factors. Of these genes, 92% were downregulated at 30 min after nitrate addition while 77% were upregulated at 3 h, suggestive of an early reprogramming of the maize RH transcriptome in response to nitrate. Predictions were made of candidate cis-acting promoter motifs underlying the distinct RH expression clusters observed. Candidate promoter motifs with a CA-rich core were the most prevalent.

### Root Hair RNA Yield

A RH growth system was designed that stimulated RH growth, offered mechanical resistance to growing roots [53] and had good gas exchange around the roots while maintaining roots in the dark. The growth system also allowed for uniform nutrient availability facilitated exchanging the nitrogen media as desired and minimized RH damage during harvesting. This system was combined with an optimized RH RNA extraction protocol (see Methods), resulting in an average yield of total RNA of 270 ng/μl from 8-12 seedlings (Bioanalyzer 2100, Agilent Technologies Inc., Santa Clara, Calif., USA).

### Determination of Nitrate Concentrations

A pilot experiment was conducted to determine limiting and optimal nitrate concentrations. Ten day old seedlings were grown in the RH growth system with Root Hair Media (RHM) containing different concentrations of nitrate (0 mM, 0.3 mM, 3 mM or 20 mM). The shoot dry biomass was highly significant between plants grown on 0.3 mM versus 3 mM nitrate (p<0.0), but not significant between 0 mM versus 0.3 mM, or between 3 mM versus 20 mM nitrate. The nutrient-rich kernel of maize was likely responsible for the modest differences in biomass observed between the different nitrogen treatments. As a result, 0.3 mM was selected as the limiting nitrogen concentration in the RH growth system, while 3 mM was defined as the optimal nitrate concentration.

### Expression Summary

After initially growing seedlings on agar with limited nitrate (0.3 mM), they were exposed to liquid media (RHM) which either maintained the 0.3 mM nitrate concentration (maintenance nitrate, MaintN) or increased it by 10-fold (3 mM, added nitrate (AddN)) with all other nutrients remaining constant. The RH transcriptome response to added nitrate at 30 min and 3 h following an extended period of nitrogen limitation was of interest. To control for possible interactions caused by adding liquid media to the seedlings, the analysis was restricted to comparing MaintN versus AddN responses within a time point. A total of 876 annotated genes (1043/46681 probes) were differentially expressed in RH between MaintN and AddN treatments at 30 min and/or 3 h. Of these genes, 256 were differentially expressed at both time points, 286 only at 30 min and 336 genes only at 3 h. Interestingly, the majority of the earlier response genes (236/256) were downregulated in response to AddN, while the majority of later response genes (259/336) were upregulated in AddN, suggestive of an early reprogramming of a portion of the RH transcriptome.

### Annotation Summary

Out of a total of 876 differentially expressed annotated genes following the addition of nitrate, 127 were putative transcription factors (15%) and another 54 were associated with phytohormones (6%). Amongst the differentially expressed genes at either and/or both 30 min and 3 h after added nitrate, several gene annotation categories were found to be overrepresented. This included the APETALA2 transcription factor family (Fisher's exact test, p=1.99e⁻⁰⁶) known to be involved in ethylene hormone responses [54]; the DOF transcription factor family (p=13.88e⁻⁰⁵), some members of which have been shown to have root-specific expression [22, 23, 55]. Other overrepresented genes were related to other hormones (primarily ABA, ethylene, auxin, jasmonic acid) (p=1.5e⁻⁰⁴); E3 RING genes involved in protein degradation (p=3.9e⁻⁰³) [56]; and genes involved in stress redox reactions (p=3e⁻⁰³) and calcium signaling (p=6.5e⁻⁰³), pathways previously shown to be involved in RH development [27, 31, 57, 58]. Additional gene categories were overrepresented, including amino acid biosynthesis (p=9.8e⁻⁰³), post-translational protein modification (p=0.012), and MAP kinases (p=0.031).

### Prediction of Cis-Acting Promoter Motifs

Cluster analysis was carried out on the differentially expressed genes in order to predict candidate cis-acting promoter motifs underlying each expression cluster. Genes in Cluster 1 showed slight expression increases after nitrate addition at 30 min and 3 h, while Clusters 4 and 5 showed decreases at both time points. The most overrepresented promoter motifs in these clusters were highly similar to the ALFIN1 motif [68, 249]. In Arabidopsis, ALFIN1 is one of three motifs that together are sufficient to activate nitrate reductase expression (nia1::GUS) by 13-fold in response to nitrate [66, 68]. In maize, ALFINs are responsible for increase grain yield [250].

Genes in Cluster 2 were highly upregulated at 30 min and 3 h after nitrate addition and contained several critical genes that regulate NO (nitric oxide) levels (non-symbiotic hemoglobin, 2-nitropane dioxygenase and polyamine biosynthesis). Cluster 2 promoters were over-represented with a motif nearly identical to the AMMORESVDCRNIA1 motif [251], previously shown to be responsible for activation of nitrate reductase (nia1) in Chlamydomonas reinhardtii in response to ammonium treatment [251]. Several motifs similar to AMMORESVDCRNIA1 were also discovered upstream of genes in Cluster 5.

Genes in Cluster 3 were moderately downregulated at 30 min and 3 h after nitrate addition and contained Roothairless 3, TOR regulon master regulator S6K, numerous regulatory genes and cell wall/vesicle transport genes. A nearly exact TGA1 motif was over-represented in the promoters of these genes. Transcription factor TGA1 is regulated by nitric oxide (NO) [252], and represses stress and defense genes [253]. The TGA1 motif can also be bound by transcription factor HY5 [254] which regulates root hair length [89] as noted earlier.

In addition to the ALFIN1 motif, Cluster 4 contained a motif similar to the ABA-regulated motif ABRECE3ZMRAB28 [255]. Another ABA-regulated motif, ABI4-1, was overrepresented in Cluster 1 [256, 257].

Genes in Cluster 6 were highly downregulated at 30 min and 3 h after nitrate addition, including CCR4-NOT, a global regulator in the TOR signaling pathway [126]. Cluster 6 promoters were over-represented with binding sites for the E2F transcription factor family, in particular motifs E2FAT and E2Fb. As noted earlier, E2F regulates the cell cycle [131] in part by interacting with the TOR/S6K pathway [24].

Several of the predicted motifs were CA-rich (ABI4-1, ALFIN1 TGA1, E2F) and appeared similar to one another, and along with the CT-rich motif (ABRECE3ZMRAB28), were similar to motifs upstream of Dof genes [82].

### Methods

### Biological Material

Maize inbred line B73 originated from the USDA Stock Center (PI 550473, North Central Regional PI Station, Ames, Iowa) was used in this study.

### Growth System Overview

In the optimized RH growth system, 30 ml of nutrient agar (see above) was added into a sterile glass test tube (Sigma Pyrex® Vista™ culture tube 25×200 mm, Sigma-Aldrich, St Louis, Mo., US) which was capped with a rubber stopper (VWR* Black Rubber Stoppers, 59580-182); the agar was left to solidify at a 90°-horizontal slant. The RH media (RHM) agar contained 0.3 mM nitrate and was adapted from previous studies (Liu et al., 2001; Liu et al., 2006). It consisted of: 3% (w/v) agar (Sigma-Aldrich A1296), 0.15 mM Ca(NO₃)₂ (Sigma Z37124), 4.85 mM CaCl₂ (Sigma 10043-52-4), 0.2 mM K₂SO₄ (Sigma 77778-80-5), 0.82 mM MgSO₄ (Sigma 10034-998), 0.1 mM FeNa-EDTA (PhytoTechnology Laboratories 15708-41-5) 9.1 μM MgCl₂ (Fisher biotech 7773-61-5), 0.034 μM NaMoO₄ (Sigma 10102-40-6), 18 μM H₃BO₃ (EMD BX0865-1), 0.08 mM Ca₅OH(PO₄)₃ (Fluka 21218), 0.2 μM CuSO₄ (Sigma C7631), 0.4 μM ZnSO₄ (Sigma 7446-20-0), pH 5.5-6.0. A single germinated seed was placed on the agar surface, 10-20 mm from the top of the tube. A home-made foam plug was used to gently press the seed against the agar, which was covered by pieces of rockwool; this system maintained the seed at the correct position, and prevented dessication, pathogen contamination and light exposure. The RHM agar contained calcium phosphate tri-basic to simulate RH growth [258, 259]. Roots grew along the agar surface: half of the RH penetrated the agar, which allowed for a uniform exchange of nutrients and mechanical resistance, while the other half of the RH grew into the air which facilitated gas exchange. Liquid RHM containing different concentrations of nitrogen were poured into the tubes at the start of the nitrogen treatment time course, since the tubes were half-empty. Since the RH grew in the air and in soft agar, harvesting caused minimal RH damage.

### Growth and Nitrate Treatments

Seeds were germinated on Whatmann paper (water saturated), and grown in the dark at about28° C. Uniformly germinated 2-3 day old seedlings were transferred to the RHM agar test tubes containing 0.3 mM nitrate (see above). All nitrate treatments were started between 9-10 am. The tubes were placed into adapted growth boxes which were then placed into growth chambers. The growth conditions consisted of a 16 h photoperiod, 30° C. day/22° C. night, with 250-300 μMol m-² s⁻¹ light [incandescent bulbs, and full spectrum fluorescent light incandescent light bulbs. Plants were watered everyday with about 1 ml of 0.3 mM nitrate RHM solution to maintain roots in a humid environment. At 8 days after transfer (time 0), 30 ml of low nitrate (MaintN, 0.3 mM) or normal nitrate (AddN, 3 mM) RHM was added to the tubes: the low nitrate RHM contained 0.15 mM Ca(NO₃)₂ and 4.85 mM CaCl₂, while the normal nitrate RHM contained 1.5 mM Ca(NO₃)₂ and 3.5 mM CaCl₂. Both solutions were supplemented with 3% (v/v) H₂O₂ as an oxygen source. The nitrate concentrations were defined in a pilot experiment (data not shown). Roots were harvested and frozen in liquid nitrogen at three time points following the nitrate shift: time 0, 30 min and 3 h. There were 8-12 plants/time point/replicate and the experiment was replicated three times in different growth chambers.

### Root Hair RNA Extraction

At harvest, long forceps were used to pull intact agar containing roots from the tubes. Root segments below the most apical lateral root node were dissected and frozen in liquid nitrogen (LiqN) individually in 1.5 ml Eppendorf tubes which were then stored at −80° C. On the day of RH RNA extraction, tubes were placed in LiqN. One by one, LiqN was poured into each tube; a tweezer pre-dipped in LiqN was used to remove the root and place it above a clean Eppendorf tube containing 200 μl TriReagent (AM9738, Ambion, USA). A second pre-chilled tweezer was then used to shave the RH; the RH remained attached to the tweezer which was then dipped into the TriReagent. One TriReagent tube was used to harvest RH RNA for every 2-3 plants. All tubes were re-frozen at −80° C. so that all RNA per replicate could be extracted simultaneously. TriReagent samples from each replicate/time point were pooled to a maximum of 800 μl which was topped up to 1 ml with TriReagent. Samples were vortexed for 30 s, incubated at root temperature (RT) for 4-6 min, then centrifuged at 12000×g for 10 min at 4° C. The aqueous phase was removed to a new tube, which was extracted with 200 μl chloroform:isoamyl alcohol (24:1) by vortexing for 15 s and incubating at RT for 3 min. Samples were centrifuged at 12000×g for 10 min at 4° C. The upper phase was transferred to new tube to which was added 250 μl isopropanol (RT) and 250 μl of high salt solution (0.8 M sodium citrate and 1.2 M sodium chloride) in order to eliminate polysaccharides and cell wall waste [260]. The samples were incubated for 15 min at RT, then centrifuged at 12000×g for 10 min at 4° C. The supernatant was carefully removed and discarded; the RNA-containing pellet was translucent and was resuspended in 500 μl RNAse-free water. Next, 400 μl of Acid-Phenol:Chloroform (AM9720, Ambion, USA) was added to each tube; samples were vortexed for 10 s, incubated for 3 min at RT, then centrifuged at 12000×g for 10 min at 4° C. The upper phase was transferred to a new tube, to which was added 500 μl isopropanol and 66 μl 3M sodium acetate pH 5.2. The samples were vortexed briefly, incubated on ice for 10 min, then 12000×g for 10 min at 4° C. The supernatant was discarded, and the pellet washed twice with 1 ml 75% ethanol with 5 min centrifugations at 12000×g at 4° C. The pellets were slightly dried and the RNA dissolved in 50 μl of RNAse-free water with a 10 min incubation at 55° C. and periodic vortexing.

### Microarray Hybridization, Analysis and Clustering

The custom Syngenta B73 Corn 46K Affymetrix array was used to hybridize RH RNA as previously described [261]. RNA from the following plant treatments were hybridized: 0.3 mM nitrate (0 min, 30 min, 3 h post-shift); 3 mM nitrate (30 min, 3 h post-shift)]. There were three biological replicates. Bioconductor [262] and R [263] were used for subsequent gene expression analysis. Expression normalization was performed using the RMA method [264]. Differential gene expression was measured using the following linear model from the Limma Package [265]. Each of the five treatments (described above) was defined as categorical variables in the linear model, defining a design matrix (X):

$$X = ( \begin{matrix}1 & 0 & 0 & 0 \\ 1 & 0 & 0 & 0 \\ 1 & 0 & 0 & 0 \\ 0 & 1 & 0 & 0 \\ 0 & 1 & 0 & 0 \\ 0 & 1 & 0 & 0 \\ 0 & 0 & 1 & 0 \\ 0 & 0 & 1 & 0 \\ 0 & 0 & 1 & 0 \\ 0 & 0 & 0 & 1 \\ 0 & 0 & 0 & 1 \\ 0 & 0 & 0 & 1\end{matrix} )$$

The linear model was adjusted using the empirical Bayesian method from the Limma Package [265]. To define the effect of nitrogen at each time point after the treatment shift, gene expression was compared between 0.3 mM nitrate versus 3 mM nitrate RHM. The potential effect of the immersion of roots in RHM solution was also determined by comparing gene expression from roots exposed to 0.3 mM nitrate at time 0 (before emergence) and at 30 min and 3 h post-emergence. For each comparison, the p-value of the Empirical Bayesian test was corrected using the Benjamini-Hochberg method [266]. The P-value was set at 0.05. Gene annotations were retrieved using MapMan (Zm_B73 file, accessed April 11) [267] and maizesequence.org [268] annotations as a starting points to link genes to the corresponding microarray probes and annotation category via a custom Perl script.

The initial Mapman annotation file had 29,142 genes, but could only identify 34% of the probes on the microarray. After the use of MaizeSequence.org annotations, gene annotations could be retrieved for 65% of probes. Annotation categories that were overrepresented were identified using the Fisher's exact test using R programming.

### Real Time Quantitative RT-PCR

Quantitative real time reverse transcription PCR (qRT-PCR) was conducted at the University of Guelph Genomics Facility using gene specific primers. Reverse transcription were conducted using MultiScribe™ Reverse Transcriptase and qPCR using PerfeCta SYBR© Green FastMix ROX™ (Quanta BioSciences, Inc., Gaithersburg, Md.). Amplification conditions were 95° C. for 3 min, followed by 40 cycles of: denaturation, 95° C. for 15 s; annealing (55° C. for Nar2.1 and 60° C. for Nrt1.1, Nrt1.2, Nrt2.1, Nrt2.2, Nrt2.3 and Tubulin) for 30 s; extension at 72° C. for 1 min. The relative expression ratio of each target gene was calculated based on real time PCR efficiency and was normalized to Zea mays alpha-tubulin-3 (Genbank EU954789.1) as previously described [269].

### Promoter Motif Prediction

The 876 differentially expressed genes were clustered using the HOPACH method [270]. Each of the six co-expressed gene lists were searched for common promoter motifs in their promoter sequences. De novo cis-acting motifs (≧6 bp) in the −200 to +1 promoter region were predicted using a Perl-based motif discovery program that was custom developed for the maize genome, called Promzea [271]. Briefly, BLAST searches using the microarray probe sequences were conducted against the full-length collection of cDNAs defined by MaizeSequence.org. Over-represented motifs in the promoter were identified using three motif discovery tools, Weeder [272], MEME (Multiple Em for Motif Elicitation) [273] and BioProspector [274]. Each motif was re-evaluated using one of the following statistical methods: the hypergeometric test or the binomial test. All significant motifs found in the search were compared to the motif databases, Athamap [275] and PLACE [276], using STAMP software [277].

### REFERENCES

6. Mackay A D, Barber S A: Comparison of root and root hair growth in solution and soil culture. Journal of Plant Nutrition 1984, 7(12):1745-1757.
7. Johnston-Monje D, Raizada M N: Integration of Biotechnologies—Plant and Endophyte Relationships: Nutrient Management. In: Comprehensive Biotechnology, Second Edition. Edited by Moo-Young M, vol. 4: Elsevier; 2011: 713-727.
8. Covitz P A, Smith L S, Long S R: Expressed Sequence Tags from a Root-Hair-Enriched Medicago truncatula cDNA Library. Plant Physiology 1998, 117(4):1325-1332.
9. Jones M A, Raymond M J, Smirnoff N: Analysis of the root-hair morphogenesis transcriptome reveals the molecular identity of six genes with roles in root-hair development in Arabidopsis. The Plant Journal 2006, 45(1):83-100.
10. Nestler J, Schütz W, Hochholdinger F: Conserved and Unique Features of the Maize (Zea mays L.) Root Hair Proteome. Journal of Proteome Research 2011, 10(5):2525-2537.
22. Yanagisawa S: Dof Domain Proteins: Plant-Specific Transcription Factors Associated with Diverse Phenomena Unique to Plants. Plant and Cell Physiology 2004, 45(4):386-391.
23. Tsujimoto-Inui Y, Naito Y, Sakurai N, Suzuki H, Sasaki R, Takahashi H, Ohtsuki N, Nakano T, Yanagisawa S, Shibata D et al: Functional genomics of the Dof transcription factor family genes in suspension-cultured cells of Arabidopsis thaliana. Plant Biotechnology 2009, 26(1):15-28.
24. Ma X M, Blenis J: Molecular mechanisms of mTOR-mediated translational control. Nature Reviews Molecular Cell Biology 2009, 10(5):307-318.
25. Gaudin A C M, McClymont S A, Holmes B M, Lyons E, Raizada M N: Novel temporal, fine-scale and growth variation phenotypes in roots of adult-stage maize (Zea mays L.) in response to low nitrogen stress. Plant, Cell & Environment 2011:no-no.
26. Gaudin A C M, McClymont S A, Raizada M N: The Nitrogen Adaptation Strategy of the Wild Teosinte Ancestor of Modern Maize, Zea mays subsp. parviglumis. Crop Science 2011, 51(6):in Press.
27. Ewens M, Leigh R A: The Effect of Nutrient Solution Composition on the Length of Root Hairs of Wheat (Triticum aestivum L.). Journal of Experimental Botany 1985, 36(5):713-724.
28. Bloch D, Monshausen G, Singer M, Gilroy S, Yalovsky S: Nitrogen source interacts with ROP signalling in root hair tip-growth. Plant, Cell & Environment 2011, 34(1):76-88.
29. Robinson D, Rorison I H: Root hairs and plant growth at low nitrogen avaibilities. New Phytologist 1987, 107(4):681-693.
30. Foehse D, Jungk A: Influence of phosphate and nitrate supply on root hair formation of rape, spinach and tomato plants. Plant and Soil 1983, 74(3):359-368.
31. Libault M, Brechenmacher L, Cheng J, Xu D, Stacey G: Root hair systems biology. Trends in Plant Science 2010, 15(11):641-650.
53. Beemster G T S, Masle J, Williamson R E, Farquhar G D: Effects of soil resistance to root penetration on leaf expansion in wheat (Triticum aestivum L.): kinematic analysis of leaf elongation. Journal of Experimental Botany 1996, 47(11):1663-1678.
54. Song C-P, Agarwal M, Ohta M, Guo Y, Halfter U, Wang P, Zhu J-K: Role of an Arabidopsis AP2/EREBP-Type Transcriptional Repressor in Abscisic Acid and Drought Stress Responses. The Plant Cell 2005, 17(8):2384-2396.
55, Yanagisawa S: Dof DNA-Binding Domains of Plant Transcription Factors Contribute to Multiple Protein-Protein Interactions. European Journal of Biochemistry 1997, 250(2):403-410.
56. Smalle J, Vierstra R D: The Ubiquitin 26S Proteasome Proteolytic Pathway. Annual Review of Plant Biology 2004, 55(1):555-590.
57. Gilroy S, Jones D L: Through form to function: root hair development and nutrient uptake. Trends in Plant Science 2000, 5(2):56-60.
58. Carol R J, Dolan L: The role of reactive oxygen species in cell growth: lessons from root hairs. Journal of Experimental Botany 2006, 57(8):1829-1834.
66. Konishi M, Yanagisawa S: The Regulatory Region Controlling the Nitrate-Responsive Expression of a Nitrate Reductase Gene, NIA1, in Arabidopsis. Plant and Cell Physiology 2011, 52(5):824-836.
67. Masclaux-Daubresse C, Daniel-Vedele F, Dechorgnat J, Chardon F, Gaufichon L, Suzuki A: Nitrogen uptake, assimilation and remobilization in plants: challenges for sustainable and productive agriculture. Annals of Botany 2010, 105(7):1141-1157.
68. Wang R, Guan P, Chen M, Xing X, Zhang Y, Crawford N M: Multiple Regulatory Elements in the Arabidopsis NIA1 Promoter Act Synergistically to Form a Nitrate Enhancer. Plant Physiology 2010, 154(1):423-432.
82. Gaur V, Singh U, Kumar A: Transcriptional profiling and in silico analysis of Dof transcription factor gene family for understanding their regulation during seed development of rice &lt;i&gt;Oryza sativa L. Molecular Biology Reports 2011, 38(4):2827-2848.
89. Oyama T, Shimura Y, Okada K: The Arabidopsis HY5 gene encodes a bZIP protein that regulates stimulus-induced development of root and hypocotyl. Genes & Development 1997, 11(22):2983-2995.
126. Collart M A: Global control of gene expression in yeast by the Ccr4-Not complex. Gene 2003, 313(0):1-16.
131. Johnson D G, Schwarz J K, Cress W D, Nevins J R: Expression of transcription factor E2F1 induces quiescent cells to enter S phase. Nature 1993, 365(6444):349-352.
249. Bastola D R, Pethe V V, Winicov I: Alfin1, a novel zinc-finger protein in alfalfa roots that binds to promoter elements in the salt-inducible MsPRP2 gene. Plant Molecular Biology 1998, 38(6):1123-1135.
250. Bruce W, Niu X: Yield enhancement in plants by modulation of maize alfins. In. United States America: CropDesign N. V.; 2010.
251. Loppes R, Radoux M: Identification of short promoter regions involved in the transcriptional expression of the nitrate reductase gene in Chlamydomonas reinhardtii. Plant Molecular Biology 2001, 45(2):215-227.
252. Lindermayr C, Sell S, Müller B, Leister D, Durner J: Redox Regulation of the NPR1-TGA1 System of Arabidopsis thaliana by Nitric Oxide. The Plant Cell 2010, 22(8):2894-2907.
253. Eckardt N A: A New Twist on Systemic Acquired Resistance: Redox Control of the NPR1-TGA1 Interaction by Salicylic Acid. The Plant Cell 2003, 15(9):1947-1949.
254. Song Y H, Yoo C M, Hong A P, Kim S H, Jeong H J, Shin S Y, Kim H J, Yun D-J, Lim C O, Bahk J D et al: DNA-Binding Study Identifies C-Box and Hybrid C/G-Box or C/A-Box Motifs as High-Affinity Binding Sites for STF1 and LONG HYPOCOTYL5 Proteins. Plant Physiology 2008, 146(4):1862-1877.
255. Busk P K, Pages M: Protein Binding to the Abscisic Acid-Responsive Element Is Independent of VIVIPAROUS1 in Vivo. The Plant Cell 1997, 9(12):2261-2270.
256. Niu X, Helentjaris T, Bate N J: Maize ABI4 Binds Coupling Element1 in Abscisic Acid and Sugar Response Genes. The Plant Cell 2002, 14(10):2565-2575.
257. Penfield S, Li Y, Gilday A D, Graham S, Graham I A: Arabidopsis ABA INSENSITIVE4 Regulates Lipid Mobilization in the Embryo and Reveals Repression of Seed Germination by the Endosperm. The Plant Cell 2006, 18(8):1887-1899.
258. Liu G, Dunlop J, Phung T: Exotically long and prolific root hairs of Arabidopsis, corn and wheat grown in a phosphate nutrient buffer system. In: Plant Nutrition, 2001: 586-587.
259. Liu G-d, James D, Thai P: Induction of Root Hair Growth in a Phosphorus-Buffered Culture Solution. Agricultural Sciences in China 2006, 5(5):370-376.
260. Chomczynski P, Mackey K: Short technical reports. Modification of the TRI reagent procedure for isolation of RNA from polysaccharide- and proteoglycan-rich sources. BioTechniques 1995, 19(6):942-945.
261. Wagner F, Radelof U: Performance of different small sample RNA amplification techniques for hybridization on Affymetrix GeneChips. Journal of Biotechnology 2007, 129(4):628-634.
262. Gentleman R, Carey V, Bates D, Bolstad B, Dettling M, Dudoit S, Ellis B, Gautier L, Ge Y, Gentry J et al: Bioconductor: open software development for computational biology and bioinformatics. Genome Biology 2004, 5(10):R80.
263. R: A Language and Environment for Statistical Computing [http://www.R-project.org]
264. Irizarry R A, Bolstad B M, Collin F, Cope L M, Hobbs B, Speed T P: Summaries of Affymetrix GeneChip probe level data. Nucleic Acids Research 2003, 31(4):e15.
265. Smyth G: limma: Linear Models for Microarray Data. In: Bioinformatics and Computational Biology Solutions Using R and Bioconductor. Edited by Gentleman R, Carey V J, Huber W, Irizarry R A, Dudoit S: Springer New York; 2005: 397-420.
266. Benjamini Y, Hochberg Y: Controlling the False Discovery Rate: A Practical and Powerful Approach to Multiple Testing. Journal of the Royal Statistical Society Series B 1995, 57:289-300.
267. Thimm O, Bläsing O, Gibon Y, Nagel A, Meyer S, Krüger P, Selbig J, Müller L A, Rhee S Y, Stitt M: mapman: a user-driven tool to display genomics data sets onto diagrams of metabolic pathways and other biological processes. The Plant Journal 2004, 37(6):914-939.
268. Schnable P S, Ware D, Fulton R S, Stein J C, Wei F, Pasternak S, Liang C, Zhang J, Fulton L, Graves T A et al: The B73 maize genome: complexity, diversity, and dynamics. Science 2009, 326(5956):1112-1115.
269. Liu J, Fanjun C, Chunliang O, Glass A D M, Yiping T, Fusuo Z, Guohua M: Root size and nitrogen-uptake activity in two maize (Zea mays) inbred lines differing in nitrogen-use efficiency. Journal of Plant Nutrition and Soil Science 2009, 172(2):230-236.
270. Van der Laan M J, Pollard K S: A new algorithm for hybrid hierarchical clustering with visualization and the bootstrap. Journal of Statistical Planning and Inference 2003, 117(2):275-303.
271. Liseron-Monfils C V, Ashlock D, McNicholas P D, Fauteux F, Strömvik M, Raizada M N: Promzea: A pipeline for discovery of regulatory motifs in maize (Zea mays L.) and its application to the anthocyanin biosynthetic pathway. Plant Physiology 2011:ready for submission.
272. Pavesi G, Zambelli F, Pesole G: Weeder H: an algorithm for finding conserved regulatory motifs and regions in homologous sequences. BMC Bioinformatics 2007, 8(1):46.
273. Bailey T L, Elkan C: Fitting a mixture model by expectation maximization to discover motifs in biopolymers. In: Proceedings of the Second International Conference on Intelligent Systems for Molecular Biology. Menlo Park, Calif.: AAAI Press; 1994: 28-36.
274. Liu X, Brutlag D, Liu J: BioProspector: discovering conserved DNA motifs in upstream regulatory regions of co-expressed genes. In: Pacific Symposium on Biocomputing 2001. Edited by Altman R B, Dunker A K, Hunter L, Klein T E; 2001: 127-138.
275. Bülow L, Engelmann S, Schindler M, Hehl R: AthaMap, integrating transcriptional and post-transcriptional data. Nucleic Acids Research 2009, 37(suppl 1):D983-D986.
276. Higo K, Ugawa Y, Iwamoto M, Korenaga T: Plant cis-acting regulatory DNA elements (PLACE) database: 1999. Nucleic Acids Research 1999, 27(1):297-300.
277. Mahony S, Benos P V: STAMP: a web tool for exploring DNA-binding motif similarities. Nucleic Acids Research 2007:253-258.
278. Wolfgruber T K, Sharma A, Schneider K L, Albert P S, Koo D-H, Shi J, Gao Z, Han F, Lee H, Xu R et al: Maize Centromere Structure and Evolution: Sequence Analysis of Centromeres 2 and 5 Reveals Dynamic Loci Shaped Primarily by Retrotransposons. PLoS Genetics 2009, 5(11):e1000743.

### Example 3

### Expression of Promoter Motifs in Transgenic Plants

A β-glucuronidase (GUS) reporter gene construct can be generated by cloning truncated promoters (i.e., minimal promoters), both wild-type and mutated variants, into a Gateway® cloning vector (Life Technologies, Grand Island, N.Y.). Using standard molecular biology techniques known in the art such as restriction enzyme digestion and ligation (See, e.g., Sambrook & Russell (2001). Molecular Cloning: A Laboratory Manual, Third Edition, Cold Spring Harbor Laboratory Press, Cold Spring Harbor, N.Y., United States of America), the truncated promoters can be constructed to comprise one or more nucleotide sequences of the invention (e.g., SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20). These recombinant promoters comprising one or more nucleotide sequences of this invention can further be constructed to comprise one or more other full or partial cis-regulatory elements. The constructs with a promoter comprising one or more of the nucleotide sequences of SEQ ID NOs:1-20 can then be operably linked to a GUS reporter. It is noted that any suitable reporter gene or a gene of interest can be operably linked to the recombinant promoter.

The construct comprising the recombinant promoter can then be stably transformed into plant cells using standard transformation procedures, such as, for example, agrobacteria-mediated transformation or particle bombardment. The resultant transgenic seedlings can be planted and tested for response to exposure to nitrate, drought and/or rehydration. The whole plant and/or selected tissues will be subjected to a standard assay for expression of the marker gene or other gene operably associated with the recombinant promoter. The expression level and pattern of expression driven by the recombinant promoter comprising one or more nucleotide sequences of SEQ ID NOs:1-20 will be compared.

The foregoing is illustrative of the present invention, and is not to be construed as limiting thereof. The invention is defined by the following claims, with equivalents of the claims to be included therein.

All publications, patent applications, patents and other references cited herein are incorporated by reference in their entireties for the teachings relevant to the sentence and/or paragraph in which the reference is presented.

That which is claimed is:

1. An isolated nucleic acid comprising a promoter having one or more nucleotide sequences selected from the group consisting of SEQ ID SEQ ID NO:2, SEQ ID NO:3, SEQ ID NO:4, SEQ ID NO:5, SEQ ID NO:6, SEQ ID NO:7, and SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, and SEQ ID NO:20, wherein the promoter modulates transcription of an operably linked polynucleotide in response to nitrate (NO₃), to drought or rehydration.

2. The nucleic acid of claim 1, wherein the promoter directs leaf-specific transcription and/or root-preferred transcription.

3. The nucleic acid of claim 1, wherein the promoter is operably linked to a polynucleotide of interest.

4. An expression cassette comprising the nucleic acid of claim 3.

5. A vector comprising the expression cassette of claim 6.

6. A plant cell comprising the nucleic acid of claim 3.

7. A plant comprising the plant cell of claim 6.

8. A method of modulating the expression a polynucleotide of interest in a plant in response to nitrate (NO₃), drought or rehydration the method comprising
introducing into a plant cell the nucleic acid of claim 3 to produce a transformed plant cell;
regenerating a transformed plant from the transformed plant cell; and
exposing the transformed plant, or a plant part, or plant cell therefrom, to NO₃, drought or rehydration.

9. The method of claim 8, wherein the promoter comprises one or more nucleotide sequences selected from the group consisting of SEQ ID NO:1, SEQ ID NO:2, SEQ ID NO:3, and any combination thereof, the plant, plant part, or plant cell is exposed to NO₃, and the expression of the polynucleotide is increased.

10. The method of claim 8, wherein the promoter comprises one or more nucleotide sequences selected from the group consisting of SEQ ID NO:4, SEQ ID NO:5, SEQ ID NO:6, SEQ ID NO:7, SEQ ID NO:8, and any combination thereof, the plant, plant part, or plant cell is exposed to NO₃, and the expression of the polynucleotide is decreased

11. The method of claim 8, wherein the promoter comprises one or more nucleotide sequences selected from the group consisting of SEQ ID NO:9, SEQ ID NO:10, SEQ ID NO:19, SEQ ID NO:20, and any combination thereof, the plant, plant part, or plant cell is exposed to drought, and the expression of the polynucleotide is increased.

12. The method of claim 8, wherein the promoter comprises one or more nucleotide sequences selected from the group consisting of SEQ ID NO:11, SEQ ID NO:12, SEQ ID NO:13, SEQ ID NO:14, SEQ ID NO:15, SEQ ID NO:16, SEQ ID NO:17, SEQ ID NO:18, and any combination thereof, the plant, plant part, or plant cell is exposed to drought, and the expression of the polynucleotide is decreased.

13. The method of claim 8, wherein the promoter comprises one or more nucleotide sequences selected from the group consisting of SEQ ID NO:9, SEQ ID NO:10, SEQ ID NO:19, SEQ ID NO:20, and any combination thereof, the plant, plant part, or plant cell is exposed to rehydration, and the expression of the polynucleotide is decreased.

14. The method of claim 8, wherein the promoter comprises one or more nucleotide sequences selected from the group consisting of SEQ ID NO:11, SEQ ID NO:12, SEQ ID NO:13, SEQ ID NO:14, SEQ ID NO:15, SEQ ID NO:16, SEQ ID NO:17, SEQ ID NO:18, and any combination thereof, the plant, plant part, or plant cell is exposed to rehydration, and the expression of the polynucleotide is increased.

15. A method of producing a plant comprising the nucleic acid of claim 3, the method comprising:
introducing into a plant cell the nucleic acid of claim 3 to produce a stably transformed plant cell; and
regenerating a stably transformed plant from the plant cell.

16. A stably transformed plant produced by the method of claim 15.

17. A seed of the plant of claim 16, wherein the genome of the seed comprises the nucleic acid of claim 3.

18. A product harvested from the plant of claim 16.

19. A processed product produced from the harvested product of claim 18.

20. A crop comprising a plurality of the plant of claim 16.


SEQUENCE LISTING (20 sequences)

SEQ ID NO: 1 - DNA, 6 bp, Artificial Sequence
  Synthetic oligonucleotide
caccgc

SEQ ID NO: 2 - DNA, 6 bp, Artificial Sequence
  Synthetic oligonucleotide
cccacc

SEQ ID NO: 3 - DNA, 6 bp, Artificial Sequence
  Synthetic oligonucleotide
gccccg

SEQ ID NO: 4 - DNA, 8 bp, Artificial Sequence
  Synthetic oligonucleotide
tggcgtgg

SEQ ID NO: 5 - DNA, 9 bp, Artificial Sequence
  Synthetic oligonucleotide
gkycycsyc

SEQ ID NO: 6 - DNA, 8 bp, Artificial Sequence
  Synthetic oligonucleotide
ccccaccc

SEQ ID NO: 7 - DNA, 6 bp, Artificial Sequence
  Synthetic oligonucleotide
cccgcc

SEQ ID NO: 8 - DNA, 10 bp, Artificial Sequence
  Synthetic oligonucleotide
ywcyytcwyy

SEQ ID NO: 9 - DNA, 6 bp, Artificial Sequence
  Synthetic oligonucleotide
cancca

SEQ ID NO: 10 - DNA, 9 bp, Artificial Sequence
  Synthetic oligonucleotide
crmwcnsac

SEQ ID NO: 11 - DNA, 8 bp, Artificial Sequence
  Synthetic oligonucleotide
gaacacgg

SEQ ID NO: 12 - DNA, 10 bp, Artificial Sequence
  Synthetic oligonucleotide
ccaacscagc

SEQ ID NO: 13 - DNA, 10 bp, Artificial Sequence
  Synthetic oligonucleotide
carcgcagca

SEQ ID NO: 14 - DNA, 8 bp, Artificial Sequence
  Synthetic oligonucleotide
atgngaaa

SEQ ID NO: 15 - DNA, 6 bp, Artificial Sequence
  Synthetic oligonucleotide
gcgaaa

SEQ ID NO: 16 - DNA, 8 bp, Artificial Sequence
  Synthetic oligonucleotide
tgcgaaac

SEQ ID NO: 17 - DNA, 10 bp, Artificial Sequence
  Synthetic oligonucleotide
acccccgctt

SEQ ID NO: 18 - DNA, 8 bp, Artificial Sequence
  Synthetic oligonucleotide
cccgcttc

SEQ ID NO: 19 - DNA, 8 bp, Artificial Sequence
  Synthetic oligonucleotide
matgcayg

SEQ ID NO: 20 - DNA, 10 bp, Artificial Sequence
  Synthetic oligonucleotide
acgtacaaac